(19) United States
(12) Patent Application Publication
(10) Pub. No.: US 2011/0301146 A1
(43) Pub. Date: Dec. 8, 2011

(54) GLYCOGEN SYNTHASE KINASE-3 BETA INHIBITORS CONTAINING 7-HYDROXY-BENZOIMIDAZOLE-4-YL-METHANONE DERIVATIVES

(75) Inventors: Yo Matsuo, Kanagawa (JP); Ryuji Ohsawa, Kanagawa (JP); Shoji Hisada, Kanagawa (JP)

(73) Assignee: ONCOTHERAPY SCIENCE, INC., Kanagawa (JP)

(21) Appl. No.: 13/129,361

(22) PCT Filed: Sep. 29, 2009
(86) PCT No.: PCT/JP2009/004975

§ 371 Date: Aug. 2, 2011

Related U.S. Application Data
(60) Provisional application No. 61/116,543, filed on Nov. 20, 2008.

(51) Int. Cl. A61K 31/454; C07D 235/08; C07D 235/18; C07D 401/12; C07D 403/12; A61P 3/10; A61K 31/4184; A61K 31/496; A61P 25/28; A61P 25/24; A61P 25/18; A61P 25/06; C07D 409/04; C07D 409/14

(52) U.S. Cl. 514/210.21; 548/304.7; 548/304.4; 548/310.7; 546/199; 548/306.1; 544/370; 544/364; 514/394; 514/322; 514/254.06; 514/253.09

(57) ABSTRACT

GSK-3beta inhibitors comprising 7-Hydroxy-benzoimidazole-4-yl-methanone Derivatives are provided. For example, the inhibitors have following general formula (I).

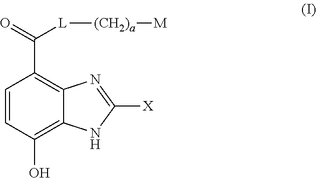

### PRIORITY

The present application claims the benefit of U.S. Provisional Application No. 61/116,543, filed on Nov. 20, 2008, the entire contents of which are incorporated by reference herein.

### TECHNICAL FIELD

The present invention relates to a compound for inhibiting GSK-3beta activity, a method for the preparation thereof, and a pharmaceutical composition containing the compound as an active ingredient.

### BACKGROUND ART

Glycogen synthase kinase-3 (GSK-3) is a proline-directed serine-threonine kinase that was initially identified as a protein which inactivates glycogen synthase through phosphorylation. Two isoforms have been identified, alpha (GSK-3alpha) and beta (GSK-3beta), which show a high degree of amino acid homology to each other. Previous studies have reported that the GSK-3beta is involved in energy metabolism, neural cell development, and body pattern formation (NPL 1).

Neurodegenerative naturopathies, including Alzheimer disease, are characterized by abnormal hyperphosphorylation of the microtubule-associated protein tau at proline-directed serine/threonine phosphorylation sites (NPL 2). GSK-3beta has been identified as a prime candidate mediating aberrant tau phosphorylation at disease-associated sites (NPLs 3-6). Hence, GSK-3beta is a promising target for therapeutic intervention in neurodegenerative tauopathies including Alzheimer disease.

Lithium carbonate, lithium citrate and lithium chloride are commonly used for the treatment of various disorders like mania, depression and migraine, and also used as an “augmenting” agent to increase the benefits of other standard drugs used for unipolar depression. Lithium is a GSK-3beta inhibitor, and therefore, GSK-3beta inhibition is a promising target for the treatment of various such disorders.

There have been reports that the activity of GSK-3 in obese diabetic mice is about twice as high as that in control (NPL 7), and the activity and expression of GSK-3 in patients with type 2 diabetes is significantly higher relativity to that in normal persons (NPL 8). Therefore, GSK-3 inhibitors are available for treatment of type 2 diabetes by reducing the activity of glucose synthase.

Taken together, GSK-3beta inhibitors can be used for a broad spectrum of diseases such as Alzheimer disease, mania, depression, migraine and type 2 diabetes and there is a strong need to develop such inhibitors for the treatment and/or prevention of GSK-3beta dependent diseases.

The present inventors have endeavored to develop an effective inhibitor of GSK-3beta and have found that a benzoimidazole derivative can selectively inhibit the activity of GSK-3beta.

### CITATION LIST

### Non Patent Literature

[NPL 1] Plyte S E, et al., Biochim. Biophys. Acta, 1114:147-162, 1992

[NPL 2] Lee V M, et al., Annu. Rev. Neurosci. 24: 1121-1159, 2001

[NPL 3] Hanger D P, et al., Neurosci. Lett. 147: 58-62, 1992

[NPL 4] Ishiguro K, et al., J. Biol. Chem. 267: 10897-10901, 1992

[NPL 5] Mandelkow E M, et al., FEBS Lett. 314: 315-321, 1992

[NPL 6] Paudel H K, et al., J. Biol. Chem. 268: 23512-23518, 1993

[NPL 7] Eldar-Finkelman H, et al., Diabetes, 48:1662-1666, 1999

[NPL 8] Nikoulina S E, et al., Diabetes, 49:263-271, 2000

### SUMMARY OF INVENTION

Accordingly, it is an object of the present invention to provide a GSK-3beta inhibitor having high inhibitory activity against GSK-3beta.

It is a further object of the present invention to provide a pharmaceutical composition including the compound, a pharmaceutically acceptable salt, hydrate, solvate, or isomer thereof for use in the treatment of GSK-3beta dependent diseases in a patient in need thereof.

In accordance with one aspect of the present invention, there is provided a compound of formula (I), and a pharmaceutically acceptable salt, hydrate, solvate, or isomer thereof:

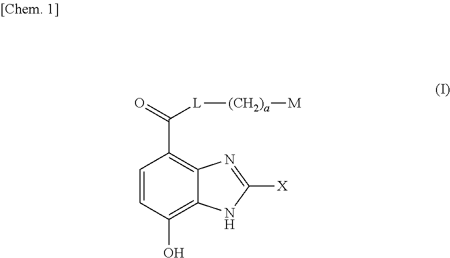

wherein

X is phenyl, thiophen-2-yl, furan-2-yl, cyclopropyl, cyclopentyl, phenylC₁-C₆ alkyl, thiophen-2-ylC₁-C₆ alkyl, furan-2-ylC₁-C₆ alkyl, cyclopropylC₁-C₆alkyl, or cyclopentylC₁-C₆ alkyl;

the said phenyl, thiophen-2-yl, furan-2-yl, cyclopropyl, cyclopentyl, phenylC₁-C₆ alkyl, thiophen-2-ylC₁-C₆ alkyl, furan-2-ylC₁-C₆ alkyl, cyclopropylC₁-C₆alkyl, or cyclopentyl C₁-C₆ alkyl are optionally substituted by 1-3 substituent(s) each independently selected from the group A;

L is —NH— or a single bond;

M is selected from C₃-C₈ cycloalkyl or 3-8 membered saturated heterocyclic group;

the C₃-C₈ cycloalkyl, and 3-8 membered saturated heterocyclic group are optionally substituted by 1-3 substituent(s) each independently selected from group A;

wherein group A consists of hydroxyl, oxo, nitro, cyano, amino, C₁-C₆ alkylamino, C₃-C₈ cycloalkylamino, amide, halogen, sulfamoyl, trifluolomethyl, p-toluenesulfonylamino, C₁-C₆ alkyl, C₃-C₈ cycloalkyl, C₁-C₆ alkoxy, C₁-C₆ alkylcarbonylamino, C₁-C₆alkylsulfonyl, C₁-C₆ alkylsulfonylamino, C₁-C₆ alkenyl, C₁-C₆ alkynyl, phosphoryl, carbonyl, carboxyl, and 3-8 membered saturated heterocyclic group; and

a is an integer from 0-5.

### DESCRIPTION OF EMBODIMENTS

Definition

In this invention, a “GSK-3beta dependent disease” is a disease in which inhibiting GSK-3beta activity is relevant to therapeutic efficacy. Such diseases include, for example, Alzheimer disease, mania, depression, migraine and type 2 diabetes. It will be understood by one of skill that such diseases do not include cancers, such as breast cancer, bladder cancer and small cell lung cancer. Thus, in some embodiments, the claimed methods of treating or preventing a GSK-3beta dependent disease exclude patients also suffering from cancer, such as breast cancer, bladder cancer or small cell lung cancer. Thus, in some embodiments, the term “a patient in need thereof” refers to a patient suffering from a GSK-3beta dependent disease, with the proviso that the patient is not also suffering from cancer, such as breast cancer, bladder cancer or small cell lung cancer.

In this invention, “alkyl” refers to a straight chain or a branched chain hydrocarbon group which does not contain any hetero atoms or unsaturated carbon-carbon bonds. “C₁-C₆alkyl” refers to an alkyl group which has 1-6 carbon atom(s). “C₁-C₄alkyl” refers to an alkyl group which has 1-4 carbon atom(s).

Examples of “C₁-C₆ alkyl” include, but are not limited to, methyl, ethyl, 1-propyl, 2-propyl, 2-methyl-1-propyl, 2-methyl-2-propyl, 1-butyl, 2-butyl, 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,3-dimethyl-1-butyl, 3,3-dimethyl-1-butyl, 2,2-dimethyl-1-butyl, 2-ethyl-1-butyl, 3,3-dimethyl-2-butyl, and 2,3-dimethyl-2-butyl.

In this invention, “phenylC₁-C₆alkyl, thiophen-2-ylC₁-C₆ alkyl, furan-2-ylC₁-C₆ alkyl, cyclopropylC₁-C₆ alkyl, or cyclopentylC₁-C₆alkyl” refers to the C₁-C₆ alkyl bound to a phenyl, thiophen-2-yl, furan-2-yl, cyclopropyl or cyclopentyl group. In one embodiment, phenylC₁-C₆ alkyl, thiophen-2-ylC₁-C₆ alkyl, furan-2-ylC₁-C₆ alkyl, cyclopropylC₁-C₆ alkyl, or cyclopentylC₁-C₆ alkyl is optionally substituted by 1-3 substituent(s) each independently selected from group A mentioned above. Such substitution may occur at either the phenyl, thiophen-2-yl, furan-2-yl, cyclopropyl, or cyclopentyl moiety or the C₁-C₆ alkyl moiety of the group, or may occur at both moieties of the group.

Examples of “phenylC₁-C₆alkyl, thiophen-2-ylC₁-C₆ alkyl, furan-2-ylC₁-C₆ alkyl, cyclopropylC₁-C₆ alkyl, or cyclopentylC₁-C₆ alkyl” include, but are not limited to, phenylmethyl, phenylethyl, phenyl-1-propyl, phenyl-2-propyl, phenyl-n-butyl, phenyl-s-butyl, phenyl-t-butyl, phenyl-2-ethylbutyl, thiophen-2-ylmethyl, thiophen-2-ylethyl, thiophen-2-yl-1-propyl, thiophen-2-yl-2-propyl, thiophen-2-yl-n-butyl, thiophen-2-yl-s-butyl, thiophen-2-yl-t-butyl, thiophen-2-yl-2-ethylbutyl, furan-2-ylmethyl, furan-2-ylethyl, furan-2-yl-1-propyl, furan-2-yl-2-propyl, furan-2-yl-n-butyl, furan-2-yl-s-butyl, furan-2-yl-t-butyl, furan-2-yl-2-ethylbutyl, cyclopropylmethyl, cyclopropylethyl, cyclopropyl-1-propyl, cyclopropyl-2-propyl, cyclopropyl-n-butyl, cyclopropyl-s-butyl, cyclopropyl-t-butyl, cyclopropyl-2-ethylbutyl, cyclopentylmethyl, cyclopentylethyl, cyclopentyl-1-propyl, cyclopentyl-2-propyl, cyclopentyl-n-butyl, cyclopentyl-s-butyl, cyclopentyl-t-butyl and cyclopentyl-2-ethylbutyl.

In this invention, “alkenyl” refers to a straight chain or a branched chain hydrocarbon group which contains one unsaturated carbon-carbon bonds and does not contain any hetero atoms. “C₁-C₆ alkenyl” refers to an alkenyl group which has 1-6 carbon atom(s).

Examples of “C₁-C₆ alkenyl”, include, but are not limited to, ethenyl, 1-propenyl, 2-propenyl, 3-propenyl, 2-methyl-prop-1-en-1-yl, 2-methyl-prop-1-en-3-yl, but-1-en-1-yl, but-1-en-2-yl, but-1-en-3-yl, but-2-en-1-yl, but-2-en-2-yl, pent-1-en-1-yl, pent-1-en-2-yl, pent-1-en-3-yl, pent-1-en-4-yl, pent-1-en-5-yl, pent-2-en-1-yl, pent-2-en-2-yl, pent-2-en-3-yl, pent-2-en-4-yl, pent-2-en-5-yl, 2-methyl-but-1-en-1-yl, 2-methyl-but-1-en-2-yl, 2-methyl-but-1-en-3-yl, 2-methyl-but-1-en-4-yl, 2-methyl-but-2-en-1-yl, 2-methyl-but-2-en-3-yl, 2-methyl-but-2-en-4-yl, 3-methyl-but-1-en-1-yl, 3-methyl-but-1-en-2-yl, 3-methyl-but-1-en-3-yl, 3-methyl-but-1-en-4-yl, 2,2-dimethyl-prop-1-en-1-yl, 2,2-dimethyl-prop-1-en-2-yl, hex-1-en-1-yl, hex-1-en-2-yl, hex-1-en-3-yl, hex-1-en-4-yl, hex-1-en-5-yl, hex-1-en-6-yl, hex-2-en-1-yl, hex-2-en-2-yl, hex-2-en-3-yl, hex-2-en-4-yl, hex-2-en-5-yl, hex-2-en-6-yl, hex-3-en-1-yl, hex-3-en-2-yl, hex-3-en-3-yl, 2-methyl-pent-1-en-1-yl, 2-methyl-pent-1-en-3-yl, 2-methyl-pent-1-en-4-yl, 2-methyl-pent-1-en-5-yl, 2-methyl-pent-2-en-1-yl, 2-methyl-pent-2-en-3-yl, 2-methyl-pent-2-en-4-yl, 2-methyl-pent-2-en-5-yl, 3-methyl-pent-1-en-1-yl, 3-methyl-pent-1-en-2-yl, 3-methyl-pent-1-en-3-yl, 3-methyl-pent-1-en-4-yl, 3-methyl-pent-1-en-5-yl, 3-methyl-pent-2-en-1-yl, 3-methyl-pent-2-en-2-yl, 3-methyl-pent-2-en-4-yl, 3-methyl-pent-2-en-5-yl, 4-methyl-pent-1-en-1-yl, 4-methyl-pent-1-en-2-yl, 4-methyl-pent-1-en-3-yl, 4-methyl-pent-1-en-4-yl, 4-methyl-pent-1-en-5-yl, 4-methyl-pent-2-en-1-yl, 4-methyl-pent-2-en-2-yl, 4-methyl-pent-2-en-3-yl, 4-methyl-pent-2-en-4-yl, 4-methyl-pent-2-en-5-yl, 2,3-dimethyl-but-1-en-1-yl, 2,3-dimethyl-but-1-en-3-yl, 2,3-dimethyl-but-1-en-4-yl, 2,3-dimethyl-but-2-en-1-yl, 3,3-dimethyl-but-1-en-1-yl, 3,3-dimethyl-but-1-en-2-yl, 3,3-dimethyl-but-1-en-4-yl, 2-ethyl-but-1-en-1-yl, 2-ethyl-but-1-en-3-yl, 2-ethyl-but-1-en-4-yl, 3-ethyl-but-1-en-1-yl, 3-ethyl-but-1-en-2-yl, 3-ethyl-but-1-en-3-yl, 3-ethyl-but-1-en-4-yl, 2-ethyl-but-2-en-1-yl, 2-ethyl-but-2-en-3-yl and 2-ethyl-but-2-en-4-yl.

In this invention, “alkynyl” refers to a straight chain or a branched chain hydrocarbon group which contains one unsaturated carbon-carbon bonds and does not contain any hetero atoms. “C₁-C₆ alkynyl” refers to an alkynyl group which has 1-6 carbon atom(s).

Examples of “C₁-C₆ alkynyl”, include, but are not limited to, ethinyl, 1-propinyl, 2-propinyl, 3-propinyl, 2-methyl-prop-1-in-1-yl, 2-methyl-prop-1-in-3-yl, but-1-in-1-yl, but-1-in-2-yl, but-1-in-3-yl, but-2-in-1-yl, but-2-in-2-yl, pent-1-in-1-yl, pent-1-in-2-yl, pent-1-in-3-yl, pent-1-in-4-yl, pent-1-in-5-yl, pent-2-in-1-yl, pent-2-in-2-yl, pent-2-in-3-yl, pent-2-in-4-yl, pent-2-in-5-yl, 2-methyl-but-1-in-1-yl, 2-methyl-but-1-in-2-yl, 2-methyl-but-1-in-3-yl, 2-methyl-but-1-in-4-yl, 2-methyl-but-2-in-1-yl, 2-methyl-but-2-in-3-yl, 2-methyl-but-2-in-4-yl, 3-methyl-but-1-in-1-yl, 3-methyl-but-1-in-2-yl, 3-methyl-but-1-in-3-yl, 3-methyl-but-1-in-4-yl, 2,2-dimethyl-prop-1-in-1-yl, 2,2-dimethyl-prop-1-in-2-yl, hex-1-in-1-yl, hex-1-in-2-yl, hex-1-in-3-yl, hex-1-in-4-yl, hex-1-in-5-yl, hex-1-in-6-yl, hex-2-in-1-yl, hex-2-in-2-yl, hex-2-in-3-yl, hex-2-in-4-yl, hex-2-in-5-yl, hex-2-in-6-yl, hex-3-in-1-yl, hex-3-in-2-yl, hex-3-in-3-yl, 2-methyl-pent-1-in-1-yl, 2-methyl-pent-1-in-3-yl, 2-methyl-pent-1-in-4-yl, 2-methyl-pent-1-in-5-yl, 2-methyl-pent-2-in-1-yl, 2-methyl-pent-2-in-3-yl, 2-methyl-pent-2-in-4-yl, 2-methyl-pent-2-in-5-yl, 3-methyl-pent-1-in-1-yl, 3-methyl-pent-1-in-2-yl, 3-methyl-pent-1-in-3-yl, 3-methyl-pent-1-in-4-yl, 3-methyl-pent-1-in-5-yl, 3-methyl-pent-2-in-1-yl, 3-methyl-pent-2-in-2-yl, 3-methyl-pent-2-in-4-yl, 3-methyl-pent-2-in-5-yl, 4-methyl-pent-1-in-1-yl, 4-methyl-pent-1-in-2-yl, 4-methyl-pent-1-in-3-yl, 4-methyl-pent-1-in-4-yl, 4-methyl-pent-1-in-5-yl, 4-methyl-pent-2-in-1-yl, 4-methyl-pent-2-in-2-yl, 4-methyl-pent-2-in-3-yl, 4-methyl-pent-2-in-4-yl, 4-methyl-pent-2-in-5-yl, 2,3-dimethyl-but-1-in-1-yl, 2,3-dimethyl-but-1-in-3-yl, 2,3-dimethyl-but-1-in-4-yl, 2,3-dimethyl-but-2-in-1-yl, 3,3-dimethyl-but-1-in-1-yl, 3,3-dimethyl-but-1-in-2-yl, 3,3-dimethyl-but-1-in-4-yl, 2-ethyl-but-1-in-1-yl, 2-ethyl-but-1-in-3-yl, 2-ethyl-but-1-in-4-yl, 3-ethyl-but-1-in-1-yl, 3-ethyl-but-1-in-2-yl, 3-ethyl-but-1-in-3-yl, 3-ethyl-but-1-in-4-yl, 2-ethyl-but-2-in-1-yl, 2-ethyl-but-2-in-3-yl and 2-ethyl-but-2-in-4-yl.

In the present invention, “alkoxy” refers to a group represented by —OR, wherein R is alkyl.

“C₁-C₆ alkoxy” refers to an alkoxy group which has 1-6 carbon atom(s). “C₁-C₄ alkoxy” refers to an alkoxy group which has 1-4 carbon atom(s).

Examples of “C₁-C₆ alkoxy” include, but are not limited to, methoxy, ethoxy, 1-propyloxy, 2-propyloxy, 2-methyl-1-propyloxy, 2-methyl-2-propyloxy, and 1-butyloxy, and 2-butyloxy.

In this invention, “C₁-C₆ alkylcarbonyl” refers to a carbonyl group bound to the C₁-C₆ alkyl. “C₁-C₄ alkylcarbonyl” refers to a carbonyl group bound to the C₁-C₄ alkyl.

Examples of “C₁-C₆ alkylcarbonyl” include, but are not limited to, methylcarbonyl, ethylcarbonyl, 1-propylcarbonyl, 2-propylcarbonyl, n-butylcarbonyl, s-butylcarbonyl, t-butylcarbonyl, and 2-ethylbutylcarbonyl.

In the present invention, “cycloalkyl” refers to a saturated carbohydrate ring system. “C₃-C₈ cycloalkyl” refers to 3-8 membered cycloalkyl.

Examples of “C₃-C₈ cycloalkyl” include, but are not limited to, cyclopropyl, cyclobutyl, cyclopentyl, cyclohexyl, cycloheptanyl, and cyclooctanyl.

In this invention, “amino” refers to a group represented by —NH₂ whose hydrogens are optionally substituted by a substituent.

In the present invention, “C₁-C₆ alkylamino” refers to an amino group bound to the C₁-C₆ alkyl.

Examples of “C₁-C₆ alkylamino” include, but are not limited to, methylamino, ethylamino, 1-propylcarbonylamino, 2-propylamino, n-butylamino, s-butylamino, t-butylamino, and 2-ethylbutylamino.

In the present invention, “C₁-C₆ alkylcarbonylamino” refers to an amino group bound to the C₁-C₆ alkylcarbonyl. “C₁-C₄ alkylcarbonylamino” refers to an amino group bound to the C₁-C₄ alkylcarbonyl.

Examples of “C₁-C₆ alkylcarbonylamino” include, but are not limited to, methylcarbonylamino, ethylcarbonylamino, 1-propylcarbonylamino, 2-propylcarbonylamino, n-butylcarbonylamino, s-butylcarbonylamino, t-butylcarbonylamino, and 2-ethylbutylcarbonylamino.

In the present invention, “C₃-C₈ cycloalkylamino” refers to an amino group bound to the C₃-C₈ cycloalkyl.

Examples of “C₃-C₈ cycloalkyl amino” include, but are not limited to, cyclopropylamino, cyclobutylamino, cyclopentylamino, cyclohexylamino, cycloheptanylamino, and cyclooctanyl amino.

In this invention, “sulfonyl” is a group represented by —SO₂—.

In this invention, “C₁-C₆ alkylsulfonyl” refers to a sulfonyl group bound to the C₁-C₆ alkyl. “C₁-C₄ alkylsulfonyl” refers to a sulfonyl group bound to the C₁-C₄ alkyl.

Examples of “C₁-C₆ alkylsulfonyl” include, but are not limited to, methylsulfonyl, ethylsulfonyl, 1-propylsulfonyl, 2-propylsulfonyl, n-butylsulfonyl, s-butylsulfonyl, t-butylsulfonyl, and 2-ethylbutylsulfonyl.

In the present invention, “C₁-C₆ alkylsulfonylamino” refers to an amino group bound to the “C₁-C₆ alkylsulfonyl”. “C₁-C₄ alkylsulfonylamino” refers to an amino group bound to the “C₁-C₄ alkylsulfonyl”.

Examples of “C₁-C₆ alkylsulfonylamino” include, but are not limited to, methylsulfonylamino, ethylsulfonylamino, 1-propylsulfonylamino, 2-propylsulfonylamino, n-butylsulfonylamino, s-butylsulfonylamino, t-butylsulfonylamino, and 2-ethylbutylsulfonylamino.

In the present invention, “a saturated heterocyclic group” refers to a saturated heterocyclic group having one or more than one hetero atom in the ring system. “3-8 membered saturated heterocyclic group” refers to a saturated heterocyclic group whose ring consists of 3-8 atoms.

Examples of “3-8 membered saturated heterocyclic group” include, but are not limited to, aziridinyl, azetidinyl, pyrrolidinyl, imidazolidinyl, piperazinyl, piperidinyl, azepanyl, and morpholinyl.

A salt is defined as the product formed from the neutralization reaction of acids and bases. Salts are ionic compounds composed of cations (positively charged ions) and anions (negative ions) so that the product is electrically neutral. These component ions can be inorganic as well as organic.

Hydrate is a term used in inorganic chemistry and organic chemistry to indicate that a substance contains water. Solvate refers to a molecule in a solution complexed by solvent molecules. Isomers are compounds with the same molecular formula but different structural formulae. More specifically, isomer includes geometric isomer, optical isomer, stereoisomer, tautomer of the compound, and mixtures thereof.

The present invention provides a compound represented by formula (I):

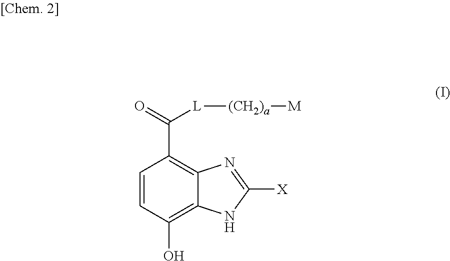

wherein

X is phenyl, thiophen-2-yl, furan-2-yl, cyclopropyl, cyclopentyl, phenylC₁-C₆alkyl, thiophen-2-ylC₁-C₆ alkyl, furan-2-ylC₁-C₆ alkyl, cyclopropylC₁-C₆alkyl, or cyclopentylC₁-C₆ alkyl;

the phenyl, thiophen-2-yl, furan-2-yl, cyclopropyl, cyclopentyl, phenylC₁-C₆alkyl, thiophen-2-ylC₁-C₆ alkyl, furan-2-ylC₁-C₆ alkyl, cyclopropylC₁-C₆alkyl, or cyclopentylC₁-C₆ alkyl are optionally substituted by 1-3 substituent(s) each independently selected from group A;

L is —NH— or single bond;

M is selected C₃-C₈ cycloalkyl or 3-8 membered saturated heterocyclic group;

the C₃-C₈ cycloalkyl, and 3-8 membered saturated heterocyclic group are optionally substituted by 1-3 substituent(s) each independently selected from group A;

wherein group A consists of hydroxyl, oxo, nitro, cyano, amino, C₁-C₆ alkylamino, C₃-C₈ cycloalkylamino, amide, halogen, sulfamoyl, trifluolomethyl, p-toluenesulfonylamino, C₁-C₆ alkyl, C₃-C₈ cycloalkyl, C₁-C₆ alkoxy, C₁-C₆ alkylcarbonylamino, C₁-C₆alkylsulfonyl, C₁-C₆ alkylsulfonylamino, C₁-C₆ alkenyl, C₁-C₆ alkynyl, phosphoryl, carbonyl, carboxyl, and 3-8 membered saturated heterocyclic group;

and a is an integer from 0-5.

Preferred compounds include those selected from the group consisting of: Example Nos. 5, 6, 7, 8, 9, 10, 11, 12, 13, 14, 15, 16, 17, 18, 19, 20, 21, 35, 36, 37, 38, 39, 40, 41, 42, 43, 44, 45, 46, 47, 48, 49, 50, 51, 52, 53, 54, 55, 56, 57, 58, 59, 60, and 72 listed in Table 1 below; and the pharmaceutically acceptable salts, prodrugs, hydrates and solvates of the forgoing compounds.

TABLE 1

| Example No. | structure | compound |
|---|---|---|
| 5 | 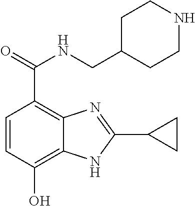 | 2-Cyclopropyl-4-hydroxy-N-(piperidin-4-ylmethyl)-1H- benzo[d]imidazole-7-carboxamide |
| 6 | 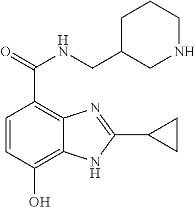 | 2-Cyclopropyl-4-hydroxy-N-(piperidin-3-yl-methyl)-1H- benzo[d]imidazole-7-carboxamide |
| 7 | 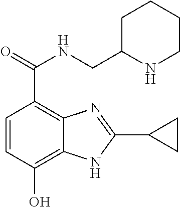 | 2-Cyclopropyl-4-hydroxy-N-(piperidin-2-ylmethyl)-1H- benzo[d]imidazole-7-carboxamide |
| 8 | 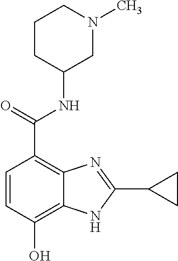 | 2-cyclopropyl-4-hydroxy-N-(1-methylpiperidin-3-yl)-1H- benzo[d]imidazole-7-carboxamide |
| 9 | 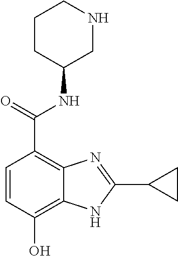 | (S)-2-cyclopropyl-4-hydroxy-N-(piperidin-3-yl)-1H-benzo [d]imidazole-7-carboxamide |
| 10 | 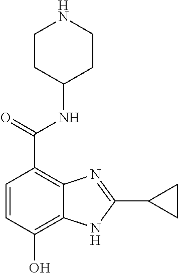 | 2-cyclopropyl-4-hydroxy-N-(piperidin-4-yl)-1H-benzo[d] imidazole-7-carboxamide |
| 11 | 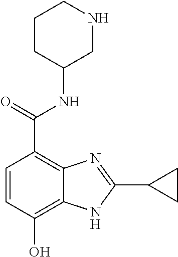 | 2-cyclopropyl-4-hydroxy-N-(piperidin-3-yl)-1H-benzo[d] imidazole-7-carboxamide |
| 12 | 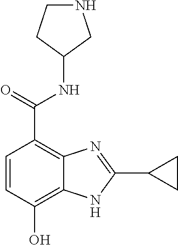 | 2-cyclopropyl-4-hydroxy-N-(pyrrolidin-3-yl)-1H-benzo[d] imidazole-7-carboxamide |
| 13 | 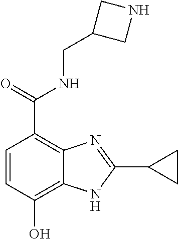 | N-(azetidin-3-ylmethyl)-2-cyclopropyl-4-hydroxy-1H- benzo[d]imidazole-7-carboxamide |
| 14 | 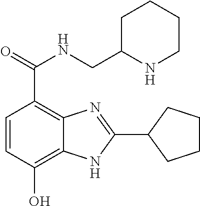 | 2-cyclopentyl-4-hydroxy-N-(piperidin-2-ylmethyl)-1H- benzo[d]imidazole-7-carboxamide |
| 15 | 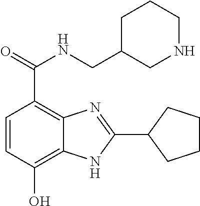 | 2-Cyclopentyl-4-hydroxy-N-(piperidin-3-ylmethyl)-1H- benzo[d]imidazole-7-carboxamide |
| 16 | 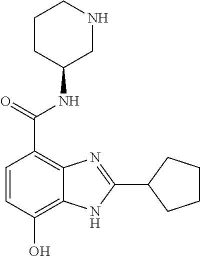 | (S)-2-Cyclopentyl-4-hydroxy-N-(piperidin-3-yl)-1H-benzo [d]imidazole-7-carboxamide |
| 17 | 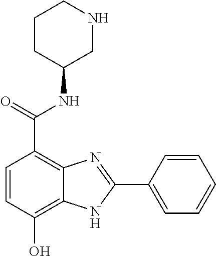 | (S)-4-Hydroxy-2-phenyl-N-(piperidin-3-yl)-1H-benzo[d] imidazole-7-carboxamide |
| 18 | 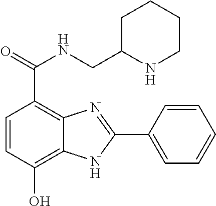 | 4-Hydroxy-2-phenyl-N-(piperidin-2-ylmethyl)-1H-benzo [d]imidazole-7-carboxamide |
| 19 | 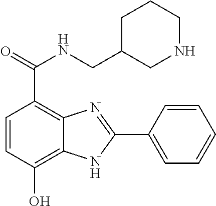 | 4-hydroxy-2-phenyl-N-(piperidin-3-ylmethyl)-1H-benzo[d] imidazole-7-carboxamide |
| 20 | 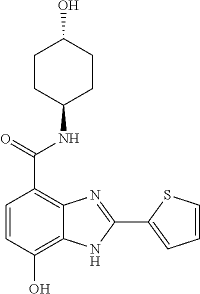 | 7-Hydroxy-N-(4-hydroxycyclohexyl)-2-(thiophen-2-yl)- 1H-benzo[d]imidazole-4-carboxamide |
| 21 | 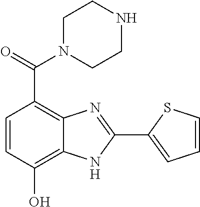 | (7-hydroxy-2-[thiophen-2-yl]-1H-benzo[d]imidazol- 4-yl)(piperazin-1-yl)methanone |
| 35 | 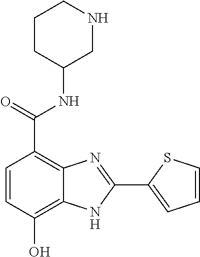 | 7-Hydroxy-N-(piperidin-3-yl)-2-(thiophen-2-yl)-1H-benzo [d]imidazole-4-carboxamide |
| 36 | 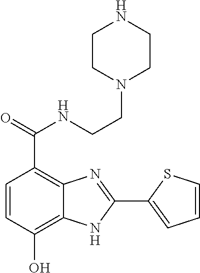 | 7-Hydroxy-N-[2-(piperazin-1-yl)ethyl]-2-(thiophen-2-yl)- 1H-benzo[d]imidazole-4-carboxamide |
| 37 | 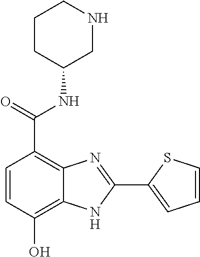 | (R)-7-Hydroxy-N-(piperidin-3-yl)-2-(thiophen-2-yl)-1H- benzo[d]imidazole-4-carboxamide |
| 38 | 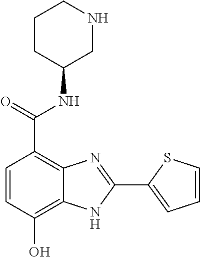 | (S)-7-Hydroxy-N-(piperidin-3-yl)-2-(thiophen-2-yl)-1H- benzo[d]imidazole-4-carboxamide |
| 39 | 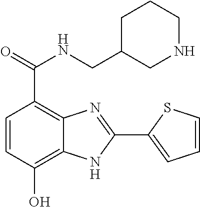 | 7-Hydroxy-N-(piperidin-3-ylmethyl)-2-(thiophen-2-yl)- 1H-benzo[d]imidazole-4-carboxamide |
| 40 | 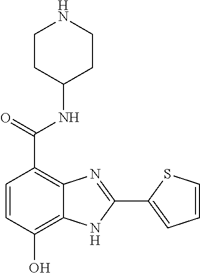 | 7-Hydroxy-N-(piperidin-4-yl)-2-(thiophen-2-yl)-1H-benzo [d]imidazole-4-carboxamide |
| 41 | 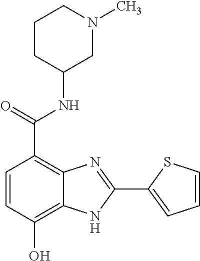 | 7-Hydroxy-N-(1-methylpiperidin-3-yl)-2-(thiophen-2-yl)- 1H-benzo[d]imidazole-4-carboxamide |
| 42 | 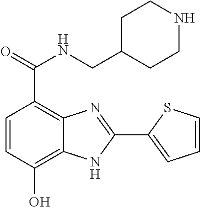 | 7-Hydroxy-N-(piperidin-4-ylmethyl)-2-(thiophen-2-yl)- 1H-benzo[d]imidazole-4-carboxamide |
| 43 | 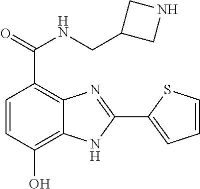 | N-(Azetidin-3-ylmethyl)-7-hydroxy-2-(thiophen-2-yl)- 1H-benzo[d]imidazole-4-carboxamide |
| 44 | 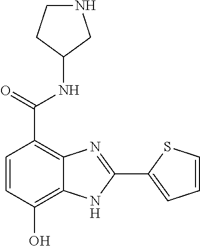 | 7-Hydroxy-N-(pyrrolidin-3-yl)-2-(thiophen-2-yl)-1H-benzo [d]imidazole-4-carboxamide |
| 45 | 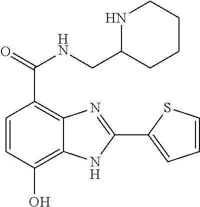 | 7-Hydroxy-N-(piperidin-2-ylmethyl)-2-(thiophen-2-yl)- 1H-benzo[d]imidazole-4-carboxamide |
| 46 | 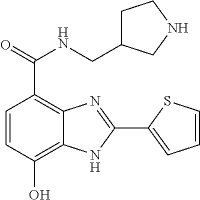 | 7-Hydroxy-N-(pyrrolidin-3-ylmethyl)-2-(thiophen-2-yl)- 1H-benzo[d]imidazole-4-carboxamide |
| 47 | 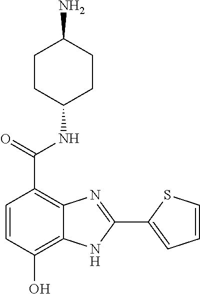 | N-(4-Aminocyclohexyl)-7-hydroxy-2-(thiophen-2-yl)-1H- benzo[d]imidazole-4-carboxamide |
| 48 | 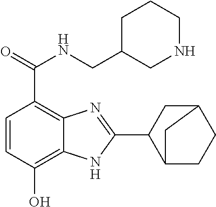 | 2-(Bicyclo[2.2.1]heptan-2-yl)-7-hydroxy-N-(piperidin-3- ylmethyl)-1H-benzo[d]imidazole-4-carboxamide |
| 49 | 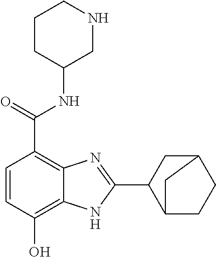 | 2-(Bicyclo[2.2.1]heptan-2-yl)-7-hydroxy-N-(piperidin-3- yl)-1H-benzo[d]imidazole-4-carboxamide |
| 50 | 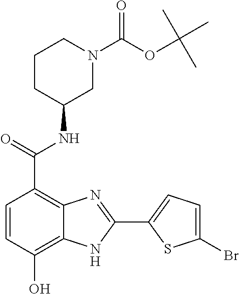 | (S)-tert-Butyl 3-(2-(5-bromothiphen-2-yl)-7-hydroxy-1H- benzo[d]imidazole-4-carboxamido)piperidine-1- carboxylate |
| 51 | 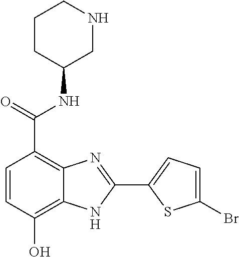 | (S)-2-(5-bromothiophen-2-yl)-7-hydroxy-N-(piperidin-3- yl)-1H-benzo[d]imidazole-4-carboxamide |
| 52 | 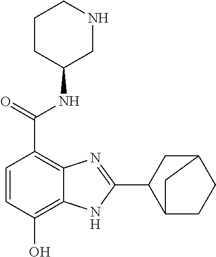 | 2-(Bicyclo[2.2.1]heptan-2-yl)-7-hydroxy-N-((S)-piperidin- 3-yl)-1H-benzo[d]imidazole-4-carboxamide |
| 53 | 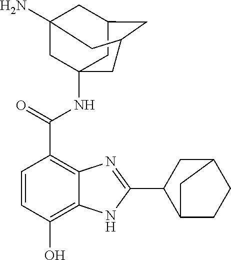 | 2-(Bicyclo[2.2.1]heptan-2-yl)-7-hydroxy-N-(adamantane- 3-ylamino)-1H-benzo[d]imidazole-4-carboxamide |
| 54 | 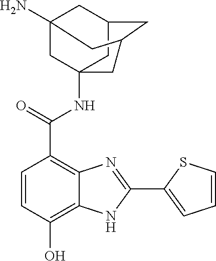 | 2-(Thiophene-2-yl)-7-hydroxy-N-(adamantane-3-ylamino)- 1H-benzo[d]imidazole-4-carboxamide |
| 55 | 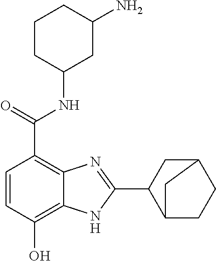 | N-(3-Aminocyclohexyl)-2-(bicyclo[2.2.1]heptan-2-yl)-7- hydroxy-1H-benzo[d]imidazole-4-carboxamide |
| 56 | 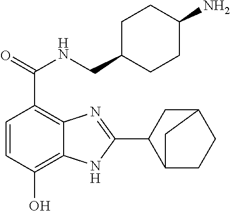 | N-{[(cis)-4-Aminocyclohexyl]methyl}-2- (bicyclo[2.2.1]heptan-2-yl)-7-hydroxy-1H- benzo[d]imidazole-4-carboxamide |
| 57 | 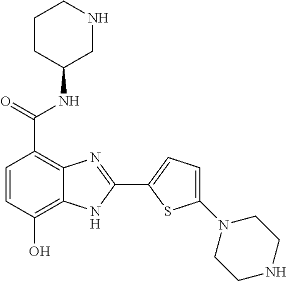 | (S)-7-hydroxy-2-(5-(piperazin-1-yl)thiophen-2-yl)-N- (piperidin-3-yl)-1H-benzo[d]imidazole-4-carboxamide |
| 58 | 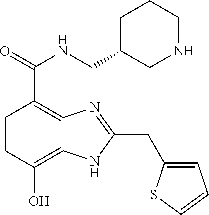 | (R)-7-hydroxy-N-(piperidin-3-ylmethyl)-2-(thiophen-2- ylmethyl)-1H-benzo[d]imidazole-4-carboxamide |
| 59 | 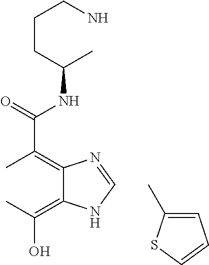 | (S)-7-hydroxy-N-(piperidin-3-yl)-2-(thiophen-2-ylmethyl)- 1H-benzo[d]imidazole-4-carboxamide |
| 60 | 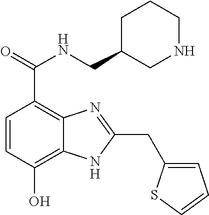 | (S)-7-hydroxy-N-(piperidin-3-ylmethyl)-2-(thiophen-2- ylmethyl)-1H-benzo[d]imidazole-4-carboxamide |
| 72 | 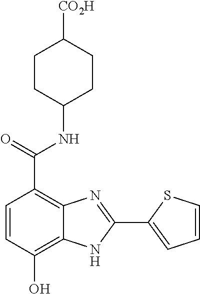 | 4-[7-hydroxy-2-(thiophen-2-yl)-1H-benzo[d]imidazole- 4-carboxamido]cyclohexanecarboxylic Acid |

The compound of formula (I) of the present invention may be in the form of a pharmaceutically acceptable salt derived from an inorganic or organic acid, and representative examples of the pharmaceutically acceptable salt derived from an inorganic or organic acid include salts obtained by adding an inorganic acid such as hydrochloric acid, hydrobromic acid, phosphoric acid or sulfonic acid, or organic carboxylic acids such as acetic acid, trifluoroacetic acid, citric acid, formic acid, maleic acid, oxalic acid, succinic acid, benzoic acid, tartaric acid, fumaric acid, mandelic acid, ascorbic acid or malic acid, methanesulfonic acid, or para toluenesulfonic acid, which do not limit its scope, to the compound of formula (I). Such acids may be prepared by the conventional processes, and other acids, which themselves are not pharmaceutically acceptable, including oxalic acid may be employed in the preparation of the bases. Alternatively, the compound of formula (I) of the present invention may also be in the form of a pharmaceutically acceptable salt derived from an inorganic or organic base include salts obtained by adding an inorganic or organic base. For example, alkalis including sodium hydroxide or potassium hydroxide, or alkaline earth metal hydroxides including calcium hydroxide, magnesium hydroxide, aluminum hydroxide or ammonium hydroxide may be used for the preparation of inorganic salt of the compound. Further, organic bases including triethylamine or diisopropylethylamine may also be used for the preparation of organic salt of the compound.

The preferred inventive compound of formula (I-II) and (I-III) may be prepared as in Scheme (I).

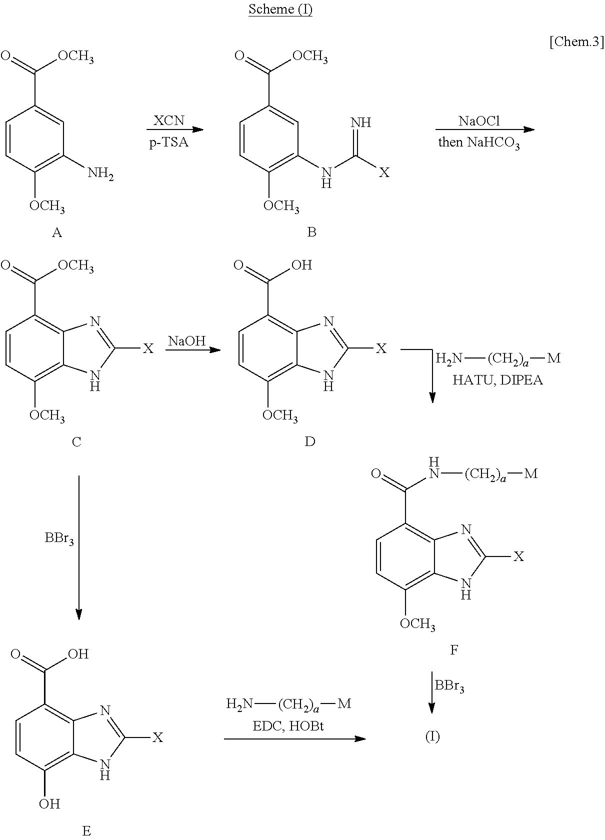

Wherein, p-TSA is p-toluenesulfonic acid, HATU is 2-(1H-7-Azabenzotriazol-1-yl)-1,1,3,3-tetramethyl uronium hexafluorophosphate Methanaminium, DIPEA is N,N-diisopropylethylamine, EDC is 1-[3-(dimethylaminopropyl)-3-ethylcarbodiimide, HOBt is 1-hydroxybenzotriazole and X, a, and M have the same meaning as defined previously.

Aniline A is reacted with the requisite nitrile in the presence of p-toluenesulfonic acid to afford amidine B. Amidine B is chlorinated with sodium hypochlorite and cyclized using sodium bicarbonate to form benzimidazole C. Intermediate C is saponified with sodium hydroxide to afford methoxy acid D which is reacted with various amines in the presence of HATU to afford amides F. Amides F are treated with boron tribromide to afford compounds of formula (I). Intermediate C is treated with boron tribromide to afford hydroxy acid E which is reacted with various amines using EDC and HOBt to afford compounds of formula (I).

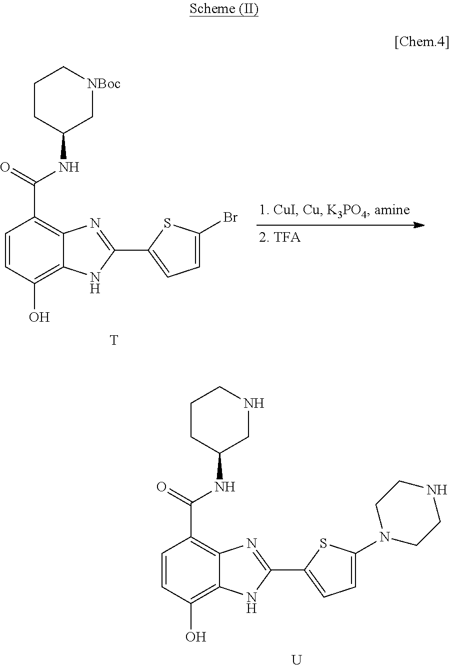

Compound T was reacted with the requisite amine in the presence of copper and copper (I) iodide followed by deprotection to afford compound U (Scheme II).

A salt, hydrate, solvate and isomer of the inventive compound of formula (I) may be prepared by employing any of the known methods. The inventive compound of formula (I), a salt, hydrate, solvate or isomer thereof may be used for the treatment of GSK-3beta dependent diseases such as Alzheimer disease, mania, depression, migraine and type 2 diabetes, by way of inhibiting GSK-3beta activity, the inventive compound having an IC₅₀ value (micro M), generally in the range of 0.0001 to 100, for example 0.001 to 50, preferably 0.001 to 10, more preferably 0.001 to 5.

Accordingly, the present invention includes a pharmaceutical composition which contains a therapeutically effective amount of the compound of formula (I), a salt, hydrate, solvate or isomer thereof as an active ingredient and a pharmaceutically acceptable carrier; therefore, the pharmaceutical composition of the present invention exerts superior preventive and treating effects on GSK-3beta dependent diseases.

A pharmaceutical formulation may be prepared in accordance with any of the conventional procedures. In preparing the formulation, the active ingredient is preferably admixed or diluted with a carrier, or enclosed within a carrier, sachet or other container. When the carrier serves as a diluent, it may be a solid, semi-solid or liquid material acting as a vehicle, excipient or medium for the active ingredient. Thus, the formulations may be in the form of a tablet, pill, powder, sachet, elixir, suspension, emulsion, solution, syrup, aerosol, soft and hard gelatin capsule, sterile injectable solution, sterile packaged powder and the like.

Examples of suitable carriers, excipients, and diluents are lactose, dextrose, sucrose, sorbitol, mannitol, calcium silicate, cellulose, methyl cellulose, microcrystalline cellulose, polyvinylpyrrolidone, water, and mineral oil. The formulations may additionally include fillers, antiemulsifiers, preservatives and the like. The compositions of the invention may be formulated so as to provide quick, sustained or delayed release of the active ingredient after their administration to a mammal by employing any of the procedures well known in the art.

The pharmaceutical composition of the present invention can be administered via various routes including oral, transdermal, subcutaneous, intravenous and intra-muscular introduction.

In addition to the above, the present composition may contain other pharmaceutical active ingredients so long as they do not inhibit the in vivo function of the compound of the present invention. For example, the composition may further contain chemotherapeutic agents conventionally used for treating Alzheimer disease, mania, depression, migraine or type 2 diabetes.

The compounds disclosed here can be used to treat or prevent GSK-3beta dependent diseases including Alzheimer disease, mania, depression, migraine and type 2 diabetes. For example, the present invention provides methods for treating or preventing GSK-3beta dependent diseases including Alzheimer disease, mania, depression, migraine and type 2 diabetes in a subject by administering to the subject the compounds disclosed here. In a preferred embodiment, such compound can be administered to the subject in the form of pharmaceutical composition including the compound of the present invention and pharmaceutically or physiologically acceptable carrier. The pharmaceutical composition of the present invention can be administered via various routes including oral, transdermal, subcutaneous, intravenous and intramuscular introduction for treating GSK-3beta dependent diseases including Alzheimer disease, mania, depression, migraine and type 2 diabetes in a subject.

In another embodiment, the present invention also provides the use of the compound of the present invention in manufacturing a pharmaceutical composition for treating GSK-3beta dependent diseases including Alzheimer disease, mania, depression, migraine and type 2 diabetes. For example, the present invention relates to a use of the compound of the present invention for manufacturing a pharmaceutical composition for treating GSK-3beta dependent diseases including Alzheimer disease, mania, depression, migraine and type 2 diabetes.

Alternatively, the present invention further provides the compound of the present invention for use in the treatment of GSK-3beta dependent diseases including Alzheimer disease, mania, depression, migraine and type 2 diabetes.

Alternatively, the present invention further provides a method or process for manufacturing a pharmaceutical composition for treating GSK-3beta dependent diseases including Alzheimer disease, mania, depression, migraine and type 2 diabetes, wherein the method or process includes the step for formulating a pharmaceutically or physiologically acceptable carrier with the compound of the present invention as active ingredients.

In another embodiment, the present invention also provides a method or process for manufacturing a pharmaceutical composition for treating GSK-3beta dependent diseases including Alzheimer disease, mania, depression, migraine and type 2 diabetes, wherein the method or process includes the step for admixing an active ingredient with a pharmaceutically or physiologically acceptable carrier, wherein the active ingredient is the compound of the present invention.

The dosage and method of administration vary according to the body-weight, age, and symptoms of the patient; however, one skilled in the art can suitably select them.

For example, although the dose of a compound of the present invention that regulates its activity depends on the symptoms, the dose is generally about 0.1 mg to about 100 mg per day, preferably about 1.0 mg to about 50 mg per day and more preferably about 1.0 mg to about 20 mg per day, when administered orally to a normal adult human (weight 60 kg).

When administering the compound parenterally, in the form of an injection to a normal adult human (weight 60 kg), although there are some differences according to the patient, target organ, symptoms and method of administration, it is convenient to intravenously inject a dose of about 0.01 mg to about 30 mg per day, preferably about 0.1 to about 20 mg per day and more preferably about 0.1 to about 10 mg per day. In the case of other animals, the appropriate dosage amount may be routinely calculated by converting to 60 kg of body-weight.

### EXAMPLES

The following examples are intended to further illustrate the present invention without limiting its scope.

### Example 1

### STEP 1: Synthesis of Methyl 4-Methoxy-3-(thiophene-2-carboximidamido)benzoate

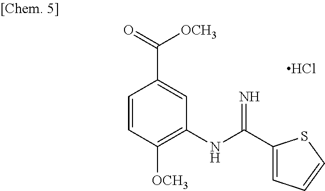

p-Toluenesulfonic acid monohydrate (42 g, 110 mmol) was heated at 120 degrees C. and once the solid completely melted, it was placed under high vacuum for 1 h to remove the water. The vacuum was released, aniline (20 g, 55 mmol) and 2-thiophenecarbonitrile (24 g, 110 mmol) were added, and the reaction mixture was heated at 160 degrees C. for 4 h. The reaction mixture was cooled to room temperature followed by addition of satd. aq NaHCO₃ (250 mL) and ethyl acetate (250 mL). The layers were separated, the aqueous layer was extracted with ethyl acetate (100 mL), and the combined organic layers were dried over Na₂SO₄, filtered, and concentrated. The crude residue was purified by column chromatography to obtain 16 g of the crude amidine intermediate. The crude intermediate was dissolved in ethyl acetate (350 mL) and HCl (2.0 M in diethyl ether, 55 mL, 110 mmol) was added. The resulting precipitate was filtered to obtain the desired product (16 g, 42% yield) as an off-white solid: ESI MS m/z 291 [C₁₄H₁₄N₃O₂S+H]⁺.

### STEP 2: Synthesis of Methyl 7-Methoxy-2-(thiophen-2-yl)-1H-benzo[d]imidazole-4-carboxylate

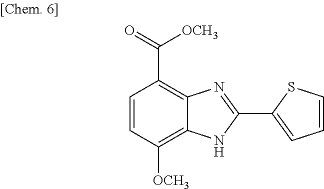

To a solution of the product from step 1 (16 g, 49 mmol) in methanol (100 mL) was added 5% aq NaOCl (75 mL, 55 mmol) and the reaction mixture was stirred at room temperature for 2 h. Next, satd. aq NaHCO₃ (150 mL) and methanol (150 mL) were added and the resulting reaction mixture was heated at 60 degrees C. for 2 d. The reaction mixture was cooled to room temperature and concentrated to remove methanol. The reaction mixture was acidified to pH 4 using 6 N HCl and the resulting precipitate was filtered and dried to obtain the desired product (8 g, 57% yield) as a brown solid: ¹H NMR (500 MHz, CDCl₃) delta 7.86 (d, J=8.5 Hz, 1H), 7.71-7.68 (m, 1H), 7.48-7.45 (m, 1H), 7.17-7.14 (m, 1H), 7.73 (d, J=8.5 Hz, 1H), 4.16 (m, 3H), 3.98 (m, 3H); ESI MS m/z 289 [C₁₄H₁₂N₂O₃S+H]⁺.

### STEP 3: Synthesis of 7-Methoxy-2-(thiophen-2-yl)-1H-benzo[d]imidazole-4-carboxylic Acid

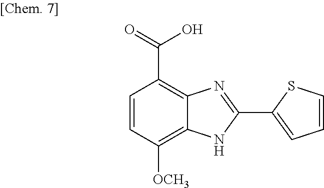

To a solution of the product from step 2 (4.2 g, 14 mmol) in ethanol (30 mL) and water (15 mL) was added 6 N NaOH (55 mL) and the reaction mixture was heated at 90 degrees C. for 2 h. The reaction mixture was cooled and concentrated to dryness. The crude residue was dissolved in water (30 ml) and acidified to pH 4 using 6 N HCl. The resulting precipitate was filtered and dried to obtain the desired product (2.2 g, 58% yield) as a brown solid: ¹H NMR (500 MHz, DMSO-d₆) delta 8.25 (d, J=3.0 Hz, 1H), 7.77 (d, J=8.0 Hz, 1H), 7.73-7.68 (m, 1H), 7.22-7.18 (m, 1H), 6.82 (d, J=8.5 Hz, 1H), 3.97 (m, 3H); ESI MS m/z 275 [C₁₃H₁₀N₂O₃S+H]⁺.

### STEP 4: Synthesis of 7-Methoxy-2-(thiophen-2-yl)-1H-benzo[d]imidazole-4-carboxylic Acid

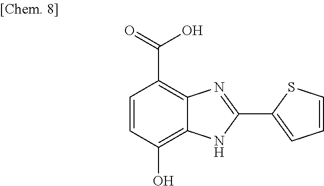

To a solution of the product from step 3 (2.5 g, 9.1 mmol) in dichloroethane (100 mL) was added BBr₃ (23 g, 91 mmol) and the reaction mixture was heated at 90 degrees C. for 2 d. The reaction mixture was cooled and poured onto ice. The resulting solids were filtered to obtain the desired product (0.45 g, 19% yield) as a brown solid. The filtrate was acidified to pH 4 using 6 N HCl and the resulting precipitate was filtered to obtain a second batch of the desired product (ALB 128328, 1.6 g, 88% yield) as a brown solid: ¹H NMR (300 MHz, CD₃OD) delta 7.93-7.90 (m, 1H), 7.75 (d, J=8.5 Hz, 1H), 7.62-7.58 (m, 1H), 7.19-7.14 (m, 1H), 6.65 (d, J=8.1 Hz, 1H); ESI MS m/z 261 [C₁₂H₈N₂O₃S+H]⁺.

### Example 2

### STEP 1: Synthesis of Methyl 3-(Cyclopropanecarboximidamido)-4-methoxybenzoate Hydrochloride

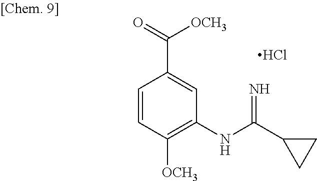

Following the procedure outlined for step 1 in Example 1, methyl 3-amino-4-methoxybenzoate (10 g, 55 mmol) was reacted with cyclopropanecarbonitrile (7.4 g, 110 mmol) to afford the desired product (16 g crude) as a black solid: ESI MS m/z 249 [C₁₃H₁₆N₂O₃+H]⁺.

### STEP 2: Synthesis of Methyl 2-Cyclopropyl-7-methoxy-1H-benzo[d]imidazole-4-carboxylate

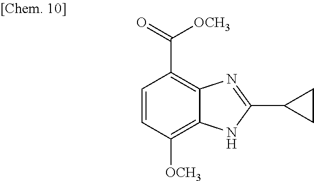

Following the procedure outlined for step 2 in Example 1, methyl 3-(cyclopropanecarboximidamido)-4-methoxybenzoate hydrochloride (15 g, 50 mmol) was reacted with aq NaOCl followed by satd. aq NaHCO₃ to afford the desired product (12 g crude) as a brown solid: ESI MS m/z 247 [C₁₃H₁₄N₂O₃+H]⁺.

### STEP 3: Synthesis of 2-Cyclopropyl-7-methoxy-1H-benzo[d]imidazole-4-carboxylic Acid

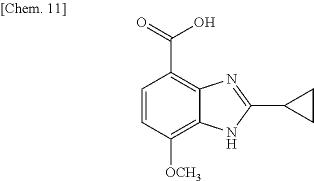

Following the procedure outlined for step 3 in Example 1, methyl 2-cyclopropyl-7-methoxy-1H-benzo[d]imidazole-4-carboxylate (2.0 g, 8.0 mmol) was reacted with sodium hydroxide to afford the desired product (1.7 g crude) as a black solid: ESI MS m/z 233 [C₁₂H₁₂N₂O₃+H]⁺.

### STEP 4: Synthesis of 2-Cyclopropyl-7-hydroxy-1H-benzo[d]imidazole-4-carboxylic Acid

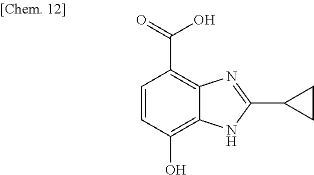

Following the procedure outlined for step 4 in Example 1, 2-cyclopropyl-7-methoxy-1H-benzo[d]imidazole-4-carboxylic acid (1.5 g, 6.1 mmol) was reacted with boron tribromide to afford the desired product (1.2 g crude) as a black solid: ESI MS m/z 219 [C₁₁H₁₀N₂O₃+H]⁺.

### Example 3

### STEP 1: Synthesis of Methyl 3-(Cyclopentanecarboximidamido)-4-methoxybenzoate Hydrochloride

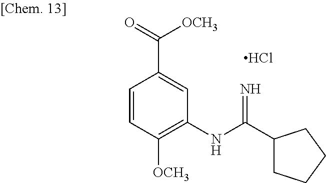

Following the procedure outlined for step 1 in Example 1, methyl 3-amino-4-methoxybenzoate (5.0 g, 27 mmol) was reacted with cyclopentanecarbonitrile (5.2 g, 55 mmol) to afford the desired product (7.7 g crude) as a brown solid: ESI MS m/z 277 [C₁₅H₂₀N₂O₃+H]⁺.

### STEP 2: Synthesis of Methyl 2-Cyclopentyl-7-methoxy-1H-benzo[d]imidazole-4-carboxylate

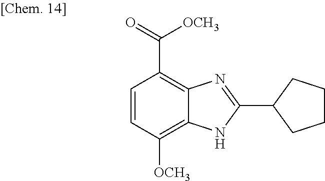

Following the procedure outlined for step 2 in Example 1, methyl 3-(cyclopentanecarboximidamido)-4-methoxybenzoate hydrochloride (5.6 g, 18 mmol) was reacted with aq NaOCl followed by satd. aq NaHCO₃ to afford the desired product (4.9 g crude) as a black solid: ESI MS m/z 275 [C₁₅H₁₈N₂O₃+H]⁺.

### STEP 3: Synthesis of 2-Cyclopentyl-7-hydroxy-1H-benzo[d]imidazole-4-carboxylic Acid

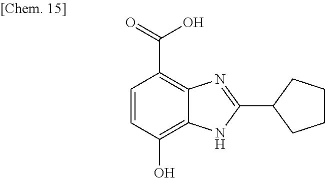

Following the procedure outlined for step 4 in Example 1, methyl 2-cyclopentyl-7-methoxy-1H-benzo[d]imidazole-4-carboxylate (1.1 g, 4.0 mmol) was reacted with boron tribromide to afford the desired product (0.92 g crude) as a black solid: ESI MS m/z 247 [C₁₃H₁₄N₂O₃+H]⁺.

### Example 4

### STEP 1: Synthesis of Methyl 3-Benzimidamido-4-methoxybenzoate Hydrochloride

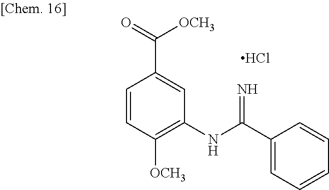

Following the procedure outlined for step 1 in Example 1, methyl 3-amino-4-methoxybenzoate (5.0 g, 27 mmol) was reacted with benzonitrile (5.7 g, 55 mmol) to afford the desired product (7.8 g crude) as a black solid: ESI MS m/z 285 [C₁₆H₁₆N₂O₃+H]⁺.

### STEP 2: Synthesis of Methyl 7-Methoxy-2-phenyl-1H-benzo[d]imidazole-4-carboxylate

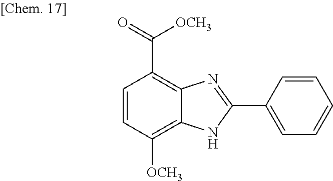

Following the procedure outlined for step 1 in Example 1, methyl 3-benzimidamido-4-methoxybenzoate hydrochloride (2.0 g, 8.0 mmol) was reacted with aq NaOCl followed by satd. aq NaHCO₃ to afford the desired product (1.7 g crude) as an off-white solid: ESI MS m/z 283 [C₁₆H₁₄N₂O₃+H]⁺.

### STEP 3: Synthesis of 7-Hydroxy-2-phenyl-1H-benzo[d]imidazole-4-carboxylic Acid

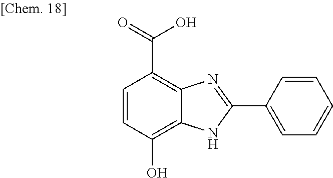

Following the procedure outlined for step 4 in Example 1, methyl 7-methoxy-2-phenyl-1H-benzo[d]imidazole-4-carboxylate (4.0 g, 12 mmol) was reacted with boron tribromide to afford the desired product (2.1 g crude) as a black solid: ESI MS m/z 255 [C₁₄H₁₀N₂O₃+H]⁺.

General Procedure A—synthesis of compounds of formula I-II as described in Scheme (1):

To a solution of hydroxy acids E (1.0 equiv) in THF (5-10 mL) was added EDC (1.2 equiv), HOBt (1.1 equiv), and the amine (1.2 equiv) and the reaction mixture was either stirred at room temperature for 16 h or heated at 50-70 degrees C. for 16 h. The reaction mixture was diluted with ethyl acetate (50 mL) and washed with water (25 ml). The layers were separated and the organic layer was dried over Na₂SO₄, concentrated, and purified by preparative HPLC (C18 silica, 10-90% acetonitrile/water with 0.05% TFA). In some instances the desired product was dissolved in trifluoroacetic acid (2 mL) and stirred for 1 h at room temperature. The reaction mixture was concentrated and eluted through an ion-exchange column (using methanol and 7 N methanol in ammonia) to obtain the desired products.

### Example 5

### 2-Cyclopropyl-4-hydroxy-N-(piperidin-4-ylmethyl)-1H-benzo[d]imidazole-7-carboxamide

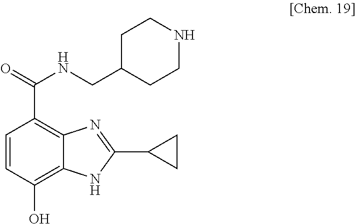

Following general procedure A, 2-cyclopropyl-4-hydroxy-1H-benzo[d]imidazole-7-carboxylic acid (55 mg, 0.25 mmol) was reacted with racemic tert-butyl 4-(aminomethyl)piperidine-1-carboxylate (81 mg, 0.38 mmol) to afford the desired product (21 mg, 27% yield) as a light brown-yellow solid: ¹H NMR (500 MHz, DMSO-d₆) delta 9.67 (bs, 1H), 7.57 (d, J=8.0 Hz, 1H), 6.58 (d, J=8.0 Hz, 1H), 3.25-3.22 (m, 2H), 2.98-2.96 (m, 2H), 2.48-2H), 2.16 (bs, 1H), 1.68-1.58 (m, 3H), 1.18-1.06 (m, 6H); ESI MS m/z 315 [C₁₇H₂₂N₄O₂+H⁺; HPLC 98.6% (AUC), t_{R}=6.38 min.

### Example 6

### 2-Cyclopropyl-4-hydroxy-N-(piperidin-3-yl-methyl)-1H-benzo[d]imidazole-7-carboxamide

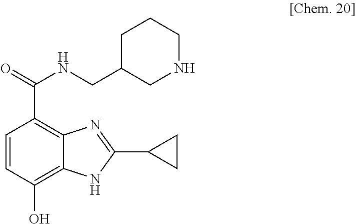

Following general procedure A, 2-cyclopropyl-4-hydroxy-1H-benzo[d]imidazole-7-carboxylic acid (55 mg, 0.25 mmol) was reacted with racemic tert-butyl 3-(aminomethyl)piperidine-1-carboxylate (81 mg, 0.38 mmol) to afford the desired product (12 mg, 15% yield) as a light gray solid: ¹H NMR (500 MHz, CD₃OD) delta 7.66 (d, J=8.0 Hz, 1H), 6.57 (d, J=8.0 Hz, 1H), 3.51-3.49 (m, 2H), 3.14 (d, J=12.5 Hz, 1H), 2.94 (bs, 1H), 2.75-2.73 (m, 1H), 2.19-2.17 (m, 1H), 1.90-1.88 (m, 2H), 1.71-1.68 (m, 2H), 1.54-1.49 (m, 1H), 1.35 (bs, 1H), 1.15-1.13 (m, 4H); ESI MS m/z 315 [C₁₇H₂₂N₄O₂+H]⁺; HPLC 99.7% (AUC), t_{R}=5.98 min.

### Example 7

### 2-Cyclopropyl-4-hydroxy-N-(piperidin-2-ylmethyl)-1H-benzo[d]imidazole-7-carboxamide

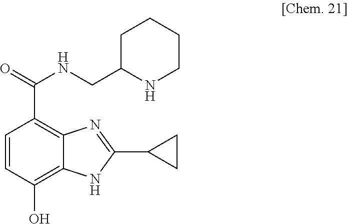

Following general procedure A, 2-cyclopropyl-4-hydroxy-1H-benzo[d]imidazole-7-carboxylic acid (55 mg, 0.25 mmol) was reacted with racemic tert-butyl 2-(aminomethyl)piperidine-1-carboxylate (81 mg, 0.38 mmol) to afford the desired product (13 mg, 17% yield) as a light gray solid: ¹H NMR (500 MHz, CD₃OD) delta 7.66 (d, J=8.0 Hz, 1H), 6.56 (d, J=8.0 Hz, 1H), 3.39-3.38 (m, 2H), 3.22 (d, J=12 Hz, 1H), 3.08 (d, J=12 Hz, 1H), 2.69-2.68 (m, 1H), 2.66-2.63 (m, 1H), 2.55-2.50 (m, 1H), 2.18-2.15 (m, 1H), 1.99-1.97 (m, 2H), 1.92-1.89 (m, 1H), 1.82-1.80 (m, 1H), 1.62-1.59 (m, 1H), 1.32-1.34 (m, 1H), 1.13 (bs, 4H); ESI MS m/z 315 [C₁₇H₂₂N₄O₂+H]⁺; HPLC 96.8% (AUC), t_{R}=6.78 min.

### Example 8

### 2-cyclopropyl-4-hydroxy-N-(1-methylpiperidin-3-yl)-1H-benzo[d]imidazole-7-carboxamide

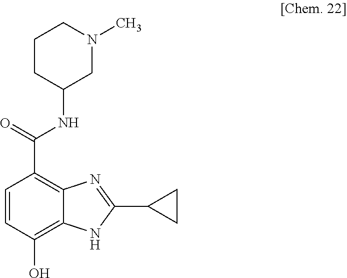

Following general procedure A, 2-cyclopropyl-4-hydroxy-1H-benzo[d]imidazole-7-carboxylic acid (55 mg, 0.25 mmol) was reacted with racemic 1-methylpiperidin-3-amine (43 mg, 0.38 mmol) to afford the desired product (21 mg, 32% yield) as a brown-yellow solid: ¹H NMR (500 MHz, DMSO-d₆) delta 12.7 (bs, 1H), 10.5 (bs, 1H), 9.97 (bs, 1H), 7.58 (d, J=8.0 Hz, 1H), 6.61 (d, J=8.0 Hz, 1H), 4.06 (bs, 1H), 3.32 (bs, 2H), 2.40-2.34 (m, 1H), 2.22-2.11 (m, 2H), 1.75 (bs, 1H), 1.65 (bs, 1H), 1.55 (bs, 1H), 1.44 (bs, 1H), 1.14-1.10 (m, 4H); ESI MS m/z 315 [C₁₇H₂₂N₄O₂+H]⁺; HPLC 96.8% (AUC), t_{R}=7.12 min.

### Example 9

### (S)-2-cyclopropyl-4-hydroxy-N-(piperidin-3-yl)-1H-benzo[d]imidazole-7-carboxamide

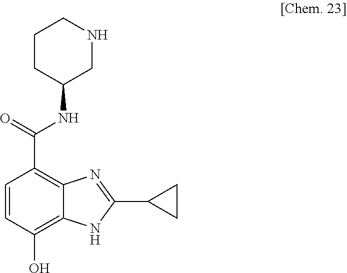

Following general procedure A, 2-cyclopropyl-4-hydroxy-1H-benzo[d]imidazole-7-carboxylic acid (55 mg, 0.25 mmol) was reacted with (S)-tert-butyl 3-aminopiperidine-1-carboxylate (75 mg, 0.38 mmol) to afford the desired product (28 mg, 37% yield) as a brown-yellow solid: ¹H NMR (500 MHz, DMSO-d₆) delta 12.7 (bs, 1H), 9.82 (bs, 1H), 7.58 (d, J=8.5 Hz, 1H), 6.61 (d, J=8.5 Hz, 1H), 3.93-3.91 (m, 1H), 3.17 (bs, 1H), 3.03-3.00(m, 1H), 2.77 (m, 1H), 2.64 (bs, 1H), 2.16 (bs, 1H), 1.87 (bs, 1H), 1.73-1.70 (m, 1H), 1.50-1.45 (m, 2H), 1.11-1.10 (m, 4H); ESI MS m/z 301 [C₁₆H₂₀N₄O₂+H]⁺; HPLC 96.8% (AUC), t_{R}=6.63 min.

### Example 10

### 2-cyclopropyl-4-hydroxy-N-(piperidin-4-yl)-1H-benzo[d]imidazole-7-carboxamide

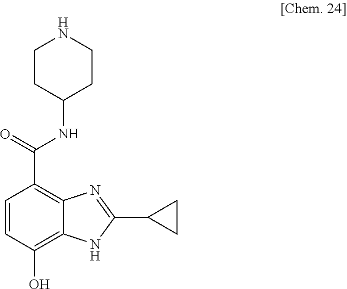

Following general procedure A, 2-cyclopropyl-4-hydroxy-1H-benzo[d]imidazole-7-carboxylic acid (55 mg, 0.25 mmol) was reacted with racemic tert-butyl 4-aminopiperidine-1-carboxylate (75 mg, 0.38 mmol) to afford the desired product (27 mg, 36% yield over two steps as a brown-yellow solid: ¹H NMR (500 MHz, DMSO-d₆) delta 9.75 (d, J=6 Hz, 1H), 7.59 (d, J=8.5 Hz, 1H), 6.61 (d, J=8.5 Hz, 1H), 3.96 (bs, 1H), 2.99-2.97 (m, 2H), 2.70-2.66 (m, 2H), 2.16 (bs, 1H), 1.88-1.86 (m, 2H), 1.42-1.40 (m, 2H), 1.13-1.04 (m, 4H); ESI MS m/z 301 [C₁₆H₂₀N₄O₂+H]⁺; HPLC 95.8% (AUC), t_{R}=6.21 min

### Example 11

### 2-cyclopropyl-4-hydroxy-N-(piperidin-3-yl)-1H-benzo[d]imidazole-7-carboxamide

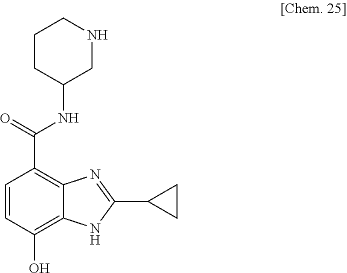

Following general procedure A, 2-cyclopropyl-4-hydroxy-1H-benzo[d]imidazole-7-carboxylic acid (55 mg, 0.25 mmol) was reacted with racemic tert-butyl 3-aminopiperidine-1-carboxylate (75 mg, 0.38 mmol) to afford the desired product (16 mg, 21% yield) as a brown-yellow solid: ¹H NMR (500 MHz, CD₃OD) delta 7.67 (d, J=8.0 Hz, 1H), 6.60 (d, J=8.0 Hz, 1H), 4.12-4.08 (m, 1H), 3.31-3.28 (m, 1H), 3.04-3.00(m, 1H), 2.79-2.73 (m, 1H), 2.20-2.10 (m, 2H), 1.94-1.91 (m, 1H), 1.76-1.65 (m, 1H), 1.17-1.14 (m, 4H); ESI MS m/z 301 [C₁₆H₂₀N₄O₂+H]⁺; HPLC 96.8% (AUC), t_{R}=6.63 min.

### Example 12

### 2-cyclopropyl-4-hydroxy-N-(pyrrolidin-3-yl)-1H-benzo[d]imidazole-7-carboxamide

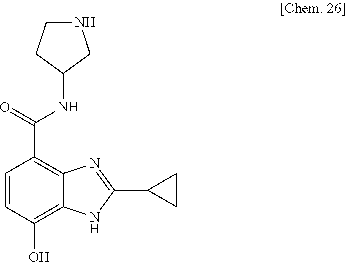

Following general procedure A, 2-cyclopropyl-4-hydroxy-1H-benzo[d]imidazole-7-carboxylic acid (55 mg, 0.25 mmol) was reacted with racemic tert-butyl 3-aminopyrrolidine-1-carboxylate (70 mg, 0.38 mmol) to afford the desired product (19 mg, 27% yield) as a brown-yellow solid: ¹H NMR (500 MHz, CD₃OD) delta 7.66 (d, J=8.0 Hz, 1H), 6.57 (d, J=8.0 Hz, 1H), 4.56-4.51 (m, 1H), 3.36-3.32 (m, 1H), 3.26-3.20(m, 1H), 3.14-3.09 (m, 1H), 3.03-3.00 (m, 1H), 2.32-2.25 (m, 1H), 2.19-2.14 (m, 1H), 1.97-1.93 (m, 1H), 1.14-1.10 (m, 4H); ESI MS m/z 287 [C₁₅H₁₈N₄O₂+H]⁺; HPLC 96.8% (AUC), t_{R}=6.40 min.

### Example 13

### N-(azetidin-3-ylmethyl)-2-cyclopropyl-4-hydroxy-1H-benzo[d]imidazole-7-carboxamide

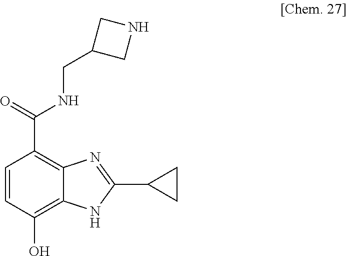

Following general procedure A, 2-cyclopropyl-4-hydroxy-1H-benzo[d]imidazole-7-carboxylic acid (55 mg, 0.25 mmol) was reacted with tert-butyl 3-(aminomethyl)azetidine-1-carboxylate (70 mg, 0.38 mmol) to afford the desired product (22 mg, 31% yield) as a brown-yellow solid: ¹H NMR (500 MHz, CD₃OD) delta 7.66 (d, J=8.5 Hz, 1H), 6.56 (d, J=8.5 Hz, 1H), 4.00-3.85 (m, 4H), 3.69-3.67 (m, 2H), 3.17-3.14(m, 1H), 2.18-2.14 (m, 1H), 1.13-1.08 (m, 4H); ESI MS m/z 287 [C₁₅H₁₈N₄O₂+H]⁺; HPLC 96.8% (AUC), t_{R}=6.15 min.

### Example 14

### Synthesis of 2-cyclopentyl-4-hydroxy-N-(piperidin-2-ylmethyl)-1H-benzo[d]imidazole-7-carboxamide

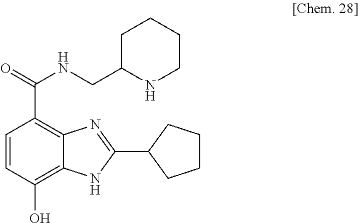

Following general procedure A, 2-cyclopentyl-4-hydroxy-1H-benzo[d]imidazole-7-carboxylic acid (62 mg, 0.25 mmol) was reacted with racemic tert-butyl 2-(aminomethyl)piperidine-1-carboxylate (81 mg, 0.38 mmol) to afford the desired product (33 mg, 39% yield) as a brown solid: ¹H NMR (300 MHz, CD₃OD) delta 7.69 (d, J=9.0 Hz, 1H), 6.59 (d, J=9.0 Hz, 1H), 3.54-3.52 (m, 2H), 3.39-3.34 (m, 1H), 3.16-3.11 (m, 1H), 2.99-2.93 (m, 1H), 2.74 (bs, 1H), 2.19-2.15 (m, 2H), 2.09-1.81 (m, 6H), 1.78-1.69 (m, 3H), 1.52-1.32 (m, 3H); ESI MS m/z 343 [C₁₉H₂₆N₄O₂+H]⁺; HPLC 98.6% (AUC), t_{R}=1.51 min.

### Example 15

### 2-Cyclopentyl-4-hydroxy-N-(piperidin-3-ylmethyl)-1H-benzo[d]imidazole-7-carboxamide

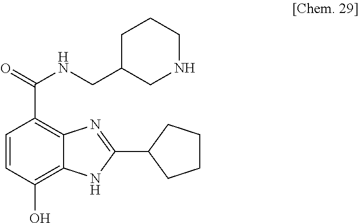

Following general procedure A, 2-cyclopentyl-4-hydroxy-1H-benzo[d]imidazole-7-carboxylic acid (62 mg, 0.25 mmol) was reacted with racemic tert-butyl 3-(aminomethyl)piperidine-1-carboxylate (81 mg, 0.38 mmol) to afford the desired product (35 mg, 41% yield) as a off-white solid: ¹H NMR (300 MHz, CD₃OD) delta 7.69 (d, J=9.0 Hz, 1H), 6.59 (d, J=9.0 Hz, 1H), 3.43-3.41 (m, 2H), 3.30-3.25 (m, 1H), 3.16-3.12 (m, 1H), 2.72-2.59 (m, 2H), 2.18-2.15 (m, 2H), 2.03-1.75 (m, 11H), 1.39-1.34 (m, 1H); ESI MS m/z 343 [C₁₉H₂₆N₄O₂+H]⁺; HPLC 99.2% (AUC), t_{R}=1.49 min.

### Example 16

### (S)-2-Cyclopentyl-4-hydroxy-N-(piperidin-3-yl)-1H-benzo[d]imidazole-7-carboxamide

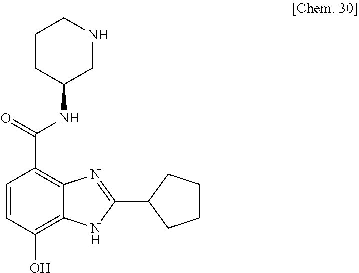

Following general procedure A, 2-cyclopentyl-4-hydroxy-1H-benzo[d]imidazole-7-carboxylic acid (55 mg, 0.25 mmol) was reacted with (S)-tert-butyl 3-aminopiperidine-1-carboxylate (75 mg, 0.38 mmol) to afford the desired product (22 mg, 27% yield) as a brown solid: ¹H NMR (500 MHz, CD₃OD) delta 7.69 (d, J=8.0 Hz, 1H), 6.61 (d, J=8.0 Hz, 1H), 4.09 (bs, 1H), 3.39-3.30 (m, 2H), 2.99 (bs, 1H), 2.72-2.76 (m, 2H), 2.19-2.14 (m, 3H), 2.03-1.99 (m, 2H), 1.91-1.88 (m, 3H), 1.78-1.67 (m, 4H); ESI MS m/z 329 [C₁₈H₂₄N₄O₂+H]⁺; HPLC >99% (AUC), t_{R}=1.48 min.

### Example 17

### (S)-4-Hydroxy-2-phenyl-N-(piperidin-3-yl)-1H-benzo[d]imidazole-7-carboxamide

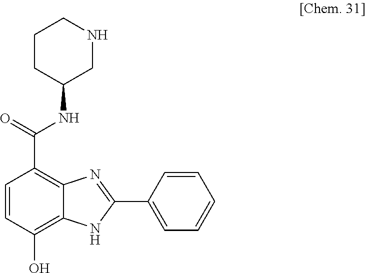

Following general procedure A, 4-hydroxy-2-phenyl-1H-benzo[d]imidazole-7-carboxylic acid (55 mg, 0.25 mmol) was reacted with (5)-tert-butyl 3-aminopiperidine-1-carboxylate (75 mg, 0.38 mmol) to afford the desired product (10 mg, 12% yield) as a green-yellow solid: ¹H NMR (500 MHz, CD₃OD) delta 8.20-8.18 (m, 2H), 7.78 (d, J=8.5 Hz, 1H), 7.56-7.51 (m, 3H), 6.68 (d, J=8.5 Hz, 1H), 4.16 (bs, 1H), 3.39-3.35(m, 1H), 3.05 (bs, 1H), 2.87-2.82 (m, 2H), 2.20-2.19 (m, 1H), 2.00 (bs, 1H), 1.80-1.76 (m, 2H); ESI MS m/z 337 [C₁₉H₂₀N₄O₂+H]⁺; HPLC 95.7% (AUC), t_{R}=8.78 min.

### Example 18

### 4-Hydroxy-2-phenyl-N-(piperidin-2-ylmethyl)-1H-benzo[d]imidazole-7-carboxamide

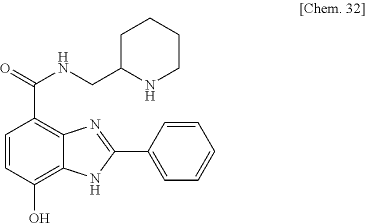

Following general procedure A, 4-hydroxy-2-phenyl-1H-benzo[d]imidazole-7-carboxylic acid (62 mg, 0.25 mmol) was reacted with racemic tert-butyl 2-(aminomethyl)piperidine-1-carboxylate (81 mg, 0.38 mmol) to afford the desired product (20 mg, 23% yield) as a brown solid: ¹H NMR (500 MHz, DMSO-d₆) delta 9.77 (bs, 1H), 8.32-8.30 (m, 2H), 7.70 (d, J=8.5 Hz, 1H), 7.58-7.51 (m, 3H), 6.71 (d, J=8.5 Hz, 1H), 3.17-3.10 (m, 2H), 2.96-2.94(m, 1H), 2.56-2.42 (m, 3H), 1.90-1.87 (m, 1H), 1.81-1.77 (m, 1H), 1.69-1.65 (m, 1H), 1.46-1.44 (m, 1H), 1.27-1.24 (m, 1H); ESI MS m/z 351 [C₂₀H₂₂N₄O₂+H]⁺; HPLC 99.0% (AUC), t_{R}=7.74 min.

### Example 19

### 4-hydroxy-2-phenyl-N-(piperidin-3-ylmethyl)-1H-benzo[d]imidazole-7-carboxamide

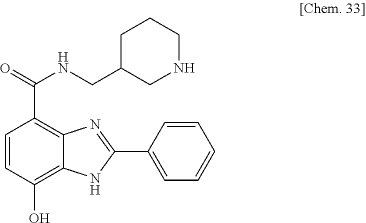

Following general procedure A, 4-hydroxy-2-phenyl-1H-benzo[d]imidazole-7-carboxylic acid (62 mg, 0.25 mmol) was reacted with tert-butyl 3-(aminomethyl)piperidine-1-carboxylate (81 mg, 0.38 mmol) to afford the desired product (20 mg, 23% yield) as a brown solid: ¹H NMR (500 MHz, DMSO-d₆) delta 9.99 (bs, 1H), 8.40-8.38 (m, 2H), 7.70 (d, J=8.5 Hz, 2H), 7.57-7.52 (m, 3H), 6.72 (d, J=8.5 Hz, 1H), 3.43-3.41 (m, 2H), 3.11-3.08(m, 1H), 2.64 (bs, 1H), 1.78-1.71 (m, 2H), 1.57 (bs, 1H), 1.69-1.65 (m, 1H), 1.46-1.44 (m, 1H), 1.27-1.24 (m, 1H); ESI MS m/z 351 [C₂₀H₂₂N₄O₂+H]⁺; HPLC 99.1 (AUC), t_{R}=8.99 min.

### Example 20

### 7-Hydroxy-N-(4-hydroxycyclohexyl)-2-(thiophen-2-yl)-1H-benzo[d]imidazole-4-carboxamide

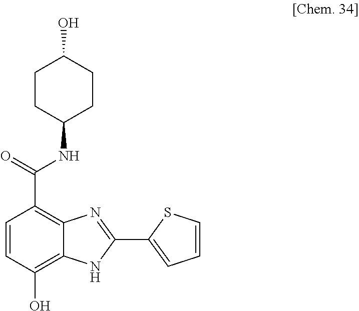

To a solution of 4-hydroxy-2-(thiophen-2-yl)-1H-benzo[d]imidazole-7-carboxylic acid (0.15 g, 0.57 mmol) in DMF (10 mL) was added HATU (0.26 g, 0.68 mmol), DIPEA (0.30 mL, 1.7 mmol) and trans-4-aminocyclohexanol (0.13 g, 1.1 mmol). The reaction mixture was heated at 50 degrees C. for 16 h. The reaction mixture was diluted with satd. aq NaHCO₃ (20 mL) and extracted with ethyl acetate (3×20 mL). The combined organic layer was dried over Na₂SO₄, concentrated, and purified by preparative HPLC (C18 silica, 10-90% acetonitrile/water with 0.05% TFA). The desired product was obtained as the trifluoroacetic acid salt which was eluted through an ion-exchange column (using methanol and 7 N methanol in ammonia) to afford the desired product (13 mg, 32%) as an off-white solid: ¹H NMR (300 MHz, CD₃OD) delta 10.19-10.17 (m, 1H), 7.87-7.85 (m, 1H), 7.79-7.75 (m, 1H), 7.64-7.61 (m, 1H), 7.22-7.19 (m, 1H), 6.71-6.67 (m, 1H), 4.02-3.97 (m, 1H), 3.77-3.71 (m, 1H), 2.19-2.08 (m, 4H), 1.55-1.50 (m, 4H); ESI MS m/z 358 [C₁₈H₁₉N₃O₃S+H]⁺; HPLC 98.8% (AUC), t_{R}=11.84 min.

### Example 21

### (7-hydroxy-2-[thiophen-2-yl]-1H-benzo[d]imidazol-4-yl)(piperazin-1-yl)methanone

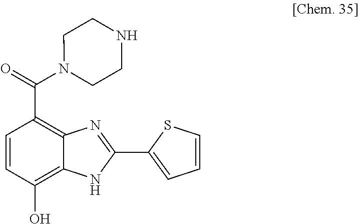

To a solution of 4-hydroxy-2-(thiophen-2-yl)-1H-benzo[d]imidazole-7-carboxylic acid (0.20 g, 0.76 mmol) in DMF (10 mL) was added HATU (0.34 g, 0.92 mmol), DIPEA (0.39 mL, 2.3 mmol) and tert-butyl piperazine-1-carboxylate (0.17 g, 0.92 mmol). The reaction mixture was heated at 50 degrees C. for 16 h. The reaction mixture was diluted with satd. aq NaHCO₃ (20 mL) and extracted with ethyl acetate (3×20 mL). The combined organic layer was dried over Na₂SO₄, concentrated, and purified by preparative HPLC (C18 silica, 10-90% acetonitrile/water with 0.05% TFA). The intermediate was dissolved in methylene dichloride and treated with 2 N HCl in ether and the reaction mixture was stirred at room temperature for 6 h. The reaction mixture was concentrated and the residue was eluted through an ion-exchange column (using methanol and 7 N methanol in ammonia) to afford the desired product (13 mg, 32%) as an off-white solid: ¹H NMR (500 MHz, DMSO-d₆) delta 8.01 (bs, 1H), 7.70 (d, J=5.0, Hz, 1H), 7.20 (dd, J=5.0, 4.0 Hz, 1H), 7.00 (d, J=8.0, Hz, 1H), 6.56-6.57 (m, 1H), 3.70-3.05 (m, 8H); ESI MS m/z 329 [C₁₆H₁₆N₄O₂S+H]⁺; HPLC 95.5% (AUC), t_{R}=8.79 min.

General Procedure B—Synthesis of Amides F as Sescribed in Scheme (1):

To a suspension of 7-methoxy-2-(thiophen-2-yl)-1H-benzo[d]imidazole-4-carboxylic acid (1.0 equiv) in toluene (5-15 mL) was added thionyl chloride (4.0 equiv). After stirring at room temperature for 16 h, the reaction mixture was heated at 70 degrees C. for 2 h. The reaction mixture was cooled, and concentrated, and the residue was suspended in THF (10-20 mL) followed by the addition of pyridine (2.0 equiv) and the corresponding amine (2.0 equiv) and the reaction mixture was heated at 70 degrees C. for 16 h. The reaction mixture was concentrated and the residue was diluted with water (20 mL) and extracted with ethyl acetate (3×20 mL). The combined organic layers were washed with satd. aq NaHCO₃ (20 mL), concentrated, and purified by flash chromatography (silica, 0-15% methanol/dichloromethane) to afford amides F. In most cases these intermediates were isolated as crude products and were carried forward without extensive characterization or further purification.

### Example 22

### tert-Butyl 3-[7-methoxy-2-(thiophen-2-yl)-1H-benzo[d]imidazole-4-carboxamido]piperidine-1-carboxylate

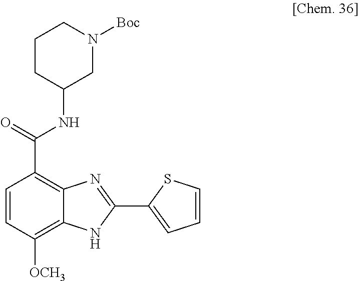

Following general procedure B, 4-methoxy-2-(thiophen-2-yl)-1H-benzo[d]imidazole-7-carboxylic acid (0.15 g, 0.44 mmol) was reacted with racemic 3-amino-1-boc-piperidine (0.18 g, 0.88 mmol) to afford the desired product (0.13 g) as a brown solid: ESI MS m/z 443 [C₂₃H₂₈N₄O₄S+H]⁺.

### Example 23

### tert-Butyl 4-{2-[7-methoxy-2-(thiophen-2-yl)-1H-benzo[d]imidazole-4-carboxamido]ethyl}piperazine-1-carboxylate

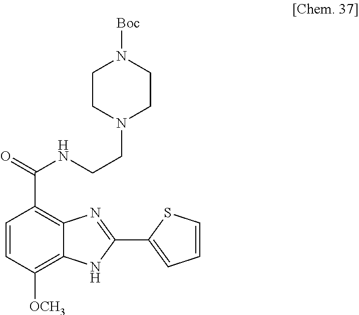

Following general procedure B, 4-methoxy-2-(thiophen-2-yl)-1H-benzo[d]imidazole-7-carboxylic acid (0.16 g, 0.58 mmol) was reacted with tert-butyl 4-(2-aminoethyl)piperazine-1-carboxylate (0.27 g, 1.2 mmol) to afford the desired product (0.24 g) as a foam: ESI MS m/z 486 [C₂₄H₃₁N₅O₄S+H]⁺.

### Example 24

### (R)-tert-Butyl 3-[7-methoxy-2-(thiophen-2-yl)-1H-benzo[d]imidazole-4-carboxamido]piperidine-1-carboxylate

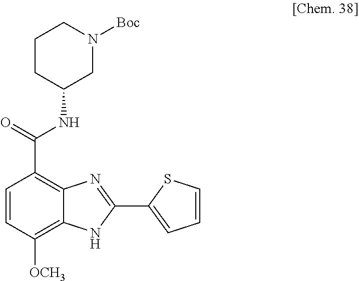

Following general procedure B, 4-methoxy-2-(thiophen-2-yl)-1H-benzo[d]imidazole-7-carboxylic acid (0.13 g, 0.46 mmol) was reacted with (R)-3-amino-1-boc-piperidine (0.18 g, 0.92 mmol) to afford the desired product (0.12 g) as a brown solid: ESI MS m/z 457 [C₂₃H₂₈N₄O₄S+H]⁺.

### Example 25

### (S)-tert-Butyl 3-[7-methoxy-2-(thiophen-2-yl)-1H-benzo[d]imidazole-4-carboxamido]piperidine-1-carboxylate

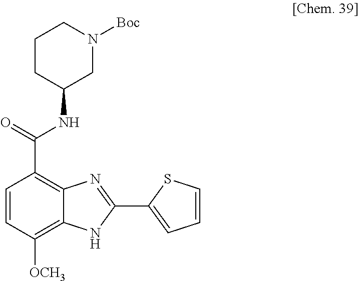

Following general procedure B, 4-methoxy-2-(thiophen-2-yl)-1H-benzo[d]imidazole-7-carboxylic acid (0.13 g, 0.46 mmol) was reacted with (S)-3-amino-1-boc-piperidine (0.18 g, 0.92 mmol) to afford the desired product (0.13 g) as a brown oil: ESI MS m/z 457 [C₂₃H₂₈N₄O₄S+H]⁺.

### Example 26

### tert-Butyl 3-{[7-methoxy-2-(thiophen-2-yl)-1H-benzo[d]imidazole-4-carboxamido]methyl}piperidine-1-carboxylate

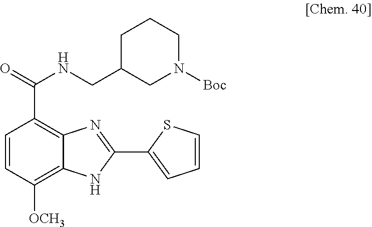

Following general procedure B, 4-methoxy-2-(thiophen-2-yl)-1H-benzo[d]imidazole-7-carboxylic acid (0.17 g, 0.62 mmol) was reacted with racemic 3-aminomethyl-1-boc-piperidine (0.26 g, 1.2 mmol) to afford the desired product (0.23 g) as a brown oil: ESI MS m/z 471 [C₂₄H₃₀N₄O₄S+H]⁺.

### Example 27

### tert-Butyl 4-[7-methoxy-2-(thiophen-2-yl)-1H-benzo[d]imidazole-4-carboxamido]piperidine-1-carboxylate

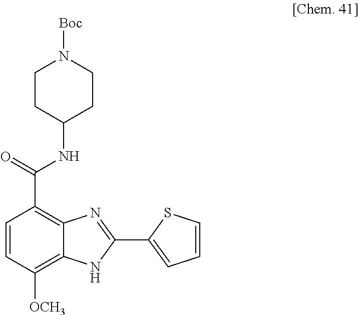

Following general procedure B, 4-methoxy-2-(thiophen-2-yl)-1H-benzo[d]imidazole-7-carboxylic acid (0.16 g, 0.58 mmol) was reacted with 4-amino-1-boc-piperidine (0.23 g, 1.2 mmol) to afford the desired product (0.20 g) as a brown oil: ESI MS m/z 457 [C₂₃H₂₈N₄O₄S+H]⁺.

### Example 28

### 7-Methoxy-N-(1-methylpiperidin-3-yl)-2-(thiophen-2-yl)-1H-benzo[d]imidazole-4-carboxamide

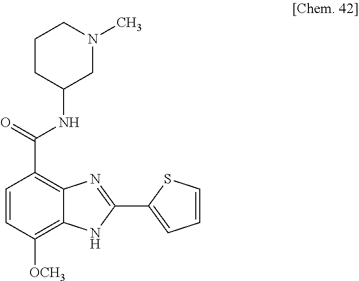

Following general procedure B, 4-methoxy-2-(thiophen-2-yl)-1H-benzo[d]imidazole-7-carboxylic acid (0.16 g, 0.59 mmol) was reacted with racemic 1-methyl-piperidin-3-amine (0.14 g, 1.2 mmol) to afford the desired product (0.15 g) as a brown glass: ESI MS m/z 371 [C₁₉H₂₂N₄O₂S+H]⁺.

### Example 29

### tert-Butyl 4-{[7-methoxy-2-(thiophen-2-yl)-1H-benzo[d]imidazole-4-carboxamido]methyl}piperidine-1-carboxylate

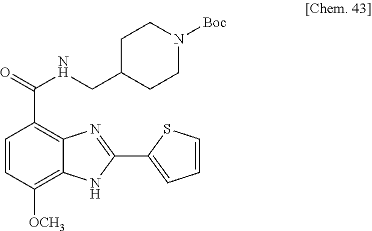

Following general procedure B, 4-methoxy-2-(thiophen-2-yl)-1H-benzo[d]imidazole-7-carboxylic acid (0.15 g, 0.56 mmol) was reacted with 4-aminomethyl-1-boc-piperidine (0.24 g, 1.1 mmol) to afford the desired product (0.16 g) as a brown foam: ESI MS m/z 471 [C₂₄H₃₀N₄O₄S+H]⁺.

### Example 30

### tert-Butyl 3-{[7-methoxy-2-(thiophen-2-yl)-1H-benzo[d]imidazole-4-carboxamido]methyl}azetidine-1-carboxylate

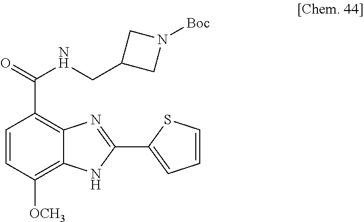

Following general procedure B, 4-methoxy-2-(thiophen-2-yl)-1H-benzo[d]imidazole-7-carboxylic acid (0.15 g, 0.56 mmol) was reacted with 1-boc-3(aminomethyl)azetidine (0.20 g, 1.1 mmol) to afford the desired product (0.17 g) as a brown foam: ESI MS m/z 443 [C₂₂H₂₆N₄O₄S+H]⁺.

### Example 31

### tert-Butyl 3-[7-methoxy-2-(thiophen-2-yl)-1H-benzo[d]imidazole-4-carboxamido]pyrrolidine-1-carboxylate

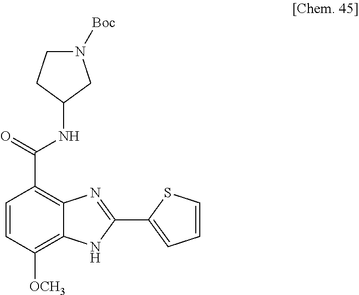

Following general procedure B, 4-methoxy-2-(thiophen-2-yl)-1H-benzo[d]imidazole-7-carboxylic acid (0.15 g, 0.56 mmol) was reacted with 3-amino-1-Boc-pyrrolidine (0.21 g, 1.1 mmol) to afford the desired product (0.20 g) as a brown oil: ESI MS m/z 443 [C₂₂H₂₆N₄O₄S+H]⁺.

### Example 32

### tert-Butyl 2-{[7-methoxy-2-(thiophen-2-yl)-1H-benzo[d]imidazole-4-carboxamido]methyl}piperidine-1-carboxylate

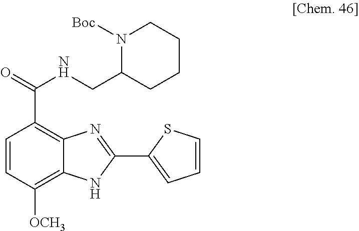

Following general procedure B, 4-methoxy-2-(thiophen-2-yl)-1H-benzo[d]imidazole-7-carboxylic acid (0.16 g, 0.58 mmol) was reacted with racemic 2-(aminomethyl)-1-N-boc-piperidine (0.25 g, 1.2 mmol) to afford the desired product (0.23 g) as a brown foam: ESI MS m/z 471 [C₂₄H₃₀N₄O₄S+H]⁺.

### Example 33

### tert-Butyl 3-{[7-methoxy-2-(thiophen-2-yl)-1H-benzo[d]imidazole-4-carboxamido]methyl}pyrrolidine-1-carboxylate

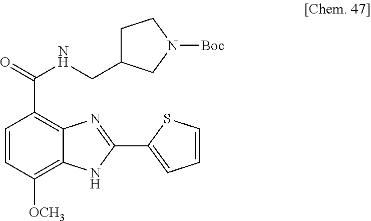

Following general procedure B, 4-methoxy-2-(thiophen-2-yl)-1H-benzo[d]imidazole-7-carboxylic acid (0.16 g, 0.58 mmol) was reacted with 3-(aminomethyl)-1-N-Boc-pyrrolidine (0.24 g, 1.2 mmol) to afford the desired product (0.19 g) as a brown oil: ESI MS m/z 457 [C₂₃H₂₈N₄O₄S+H]⁺.

### Example 34

### tert-Butyl-4-[7-methoxy-2-(thiophen-2-yl)-1H-benzo[d]imidazole-4-carboxamido]cyclohexylcarbamate

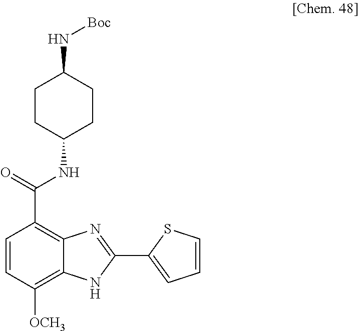

Following general procedure B, 4-methoxy-2-(thiophen-2-yl)-1H-benzo[d]imidazole-7-carboxylic acid (0.15 g, 0.55 mmol) was reacted with 1-Boc-amino-1,4-cyclohexyldiamine (0.23 g, 1.1 mmol) to afford the desired product (92 mg) as a brown oil: ESI MS m/z 471 [C₂₄H₃₀N₄O₄S+H]⁺.

General Procedure C—Synthesis of Compounds as Described in Scheme (1):

To a suspension of amides F (1.0 equiv) in dichloroethane (10-25 mL) was added boron tribromide (6.0-10 equiv) and the reaction mixture was heated at 80 degrees C. for 16 h. The reaction mixture was poured over ice and the resulting mixture was concentrated. The crude residue was eluted through an ion-exchange column (using methanol and 7 N methanol in ammonia) as a crude purification. The crude product was further purified by preparative HPLC (C18 silica, 10-90% acetonitrile/water with 0.05% TFA). The desired product was obtained as the trifluoroacetic acid salt which was eluted through an ion-exchange column (using methanol and 7 N methanol in ammonia) to obtain the desired products.

### Example 35

### 7-Hydroxy-N-(piperidin-3-yl)-2-(thiophen-2-yl)-1H-benzo[d]imidazole-4-carboxamide

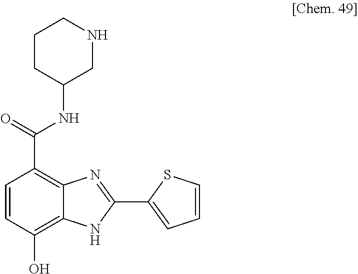

Following general procedure C, tert-Butyl 3-[7-methoxy-2-(thiophen-2-yl)-1H-benzo[d]imidazole-4-carboxamido]piperidine-1-carboxylate (0.13 g) was reacted with boron tribromide to afford the desired product (34 mg, 23% yield) as a light yellow solid: ¹H NMR (300 MHz, CD₃OD) delta 7.86-7.85 (m, 1H), 7.76 (d, J=8.4 Hz, 1H), 7.63-7.61 (m, 1H), 7.22-7.19 (m, 1H), 6.66 (d, J=8.4 Hz, 1H), 4.14-4.10 (m, 1H), 3.04-3.00 (m, 1H), 2.86-2.77 (m, 2H), 2.18-1.99 (m, 2H), 1.79-1.72 (m, 2H); ESI MS m/z 343 [C₁₇H₁₈N₄O₂S+H]⁺; HPLC 99.2% (AUC) t_{R}=9.73 min.

### Example 36

### 7-Hydroxy-N-[2-(piperazin-1-yl)ethyl]-2-(thiophen-2-yl)-1H-benzo[d]imidazole-4-carboxamide

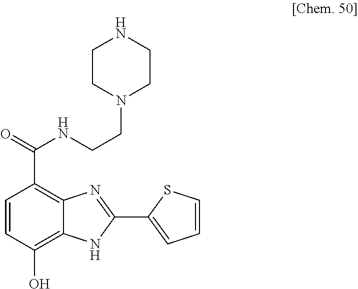

Following general procedure C, tert-Butyl 4-{2-[7-methoxy-2-(thiophen-2-yl)-1H-benzo[d]imidazole-4-carboxamido]ethyl}piperazine-1-carboxylate (0.24 g) was reacted with boron tribromide to afford the desired product (70 mg, 32% yield) as a white solid: ¹H NMR (500 MHz, DMSO-d₆) delta 9.50 (s, 1H), 8.08 (d, J=2.0 Hz, 1H), 7.77 (d, J=5.0 Hz, 1H), 7.69 (d, J=8.0 Hz, 1H), 7.25-7.24 (m, 1H), 6.71 (d, J=8.0 Hz, 1H), 3.55-3.51 (m, 3H), 2.90-2.84 (m, 5H), 2.56-2.50 (m, 3H); ESI MS m/z 372 [C₁₈H₂₁N₅O₂S+H]⁺; HPLC >99% (AUC), t_{R}=8.74 min.

### Example 37

### (R)-7-Hydroxy-N-(piperidin-3-yl)-2-(thiophen-2-yl)-1H-benzo[d]imidazole-4-carboxamide

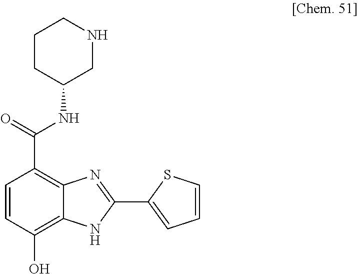

Following general procedure C, (R)-tert-Butyl 3-[7-methoxy-2-(thiophen-2-yl)-1H-benzo[d]imidazole-4-carboxamido]piperidine-1-carboxylate (0.12 g) was reacted with boron tribromide to afford the desired product (25 mg, 16% yield) as a light yellow solid: ¹H NMR (300 MHz, CD₃OD) delta 7.88-7.87 (m, 1H), 7.79-7.75 (m, 1H), 7.65-7.63 (m, 1H), 7.24-7.21 (m, 1H), 6.70-6.67 (m, 1H), 4.17-4.14 (m, 1H), 3.08-3.00 (m, 1H), 2.89-2.78 (m, 2H), 2.24-1.98 (m, 2H), 1.82-1.76 (m, 2H); ESI MS m/z 343 [C₁₇H₁₈N₄O₂S+H]⁺; HPLC 96.1% (AUC), t_{R}=10.5 min.

### Example 38

### (S)-7-Hydroxy-N-(piperidin-3-yl)-2-(thiophen-2-yl)-1H-benzo[d]imidazole-4-carboxamide

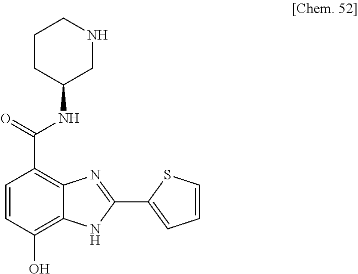

Following general procedure C, (S)-tert-Butyl 3-[7-methoxy-2-(thiophen-2-yl)-1H-benzo[d]imidazole-4-carboxamido]piperidine-1-carboxylate (0.13 g) was reacted with boron tribromide to afford the desired product (45 mg, 29% yield) as a light yellow solid: ¹H NMR (300 MHz, CD₃OD) delta 7.88-7.87 (m, 1H), 7.79-7.75 (m, 1H), 7.65-7.63 (m, 1H), 7.24-7.21 (m, 1H), 6.70-6.66 (m, 1H), 4.17-4.14 (m, 1H), 3.08-3.00 (m, 1H), 2.89-2.78 (m, 2H), 2.24-1.98 (m, 2H), 1.82-1.76 (m, 2H); ESI MS m/z 343 [C₁₇H₁₈N₄O₂S+H]⁺; HPLC >99% (AUC), t_{R}=9.80 min.

### Example 39

### 7-Hydroxy-N-(piperidin-3-ylmethyl)-2-(thiophen-2-yl)-1H-benzo[d]imidazole-4-carboxamide

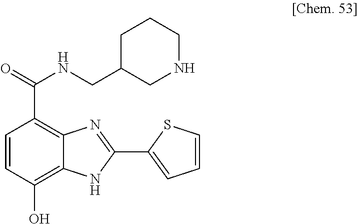

Following general procedure C, tert-Butyl 3-{[7-methoxy-2-(thiophen-2-yl)-1H-benzo[d]imidazole-4-carboxamido]methyl}piperidine-1-carboxylate (0.23 g) was reacted with boron tribromide to afford the desired product (90 mg, 41% yield) as a light brown solid: ¹H NMR (300 MHz, DMSO-d₆) delta 9.62 (s, 1H), 8.06-8.04 (m, 1H), 7.74 (d, J=4.8 Hz, 1H), 7.64 (d, J=8.4 Hz, 1H), 7.24-7.21 (m, 1H), 6.66 (d, J=8.4 Hz, 1H), 3.29 (t, J=6.0 Hz, 2H), 3.17-3.10 (m, 1H), 2.93-2.89 (m, 1H), 2.47-2.37 (m, 2H), 1.95-1.90 (m, 1H), 1.76-1.63 (m, 2H), 1.49-1.20 (m, 2H); ESI MS m/z 357 [C₁₈H₂₀N₄O₂S+H]⁺; HPLC >99% (AUC), t_{R}=9.41 min.

### Example 40

### 7-Hydroxy-N-(piperidin-4-yl)-2-(thiophen-2-yl)-1H-benzo[d]imidazole-4-carboxamide

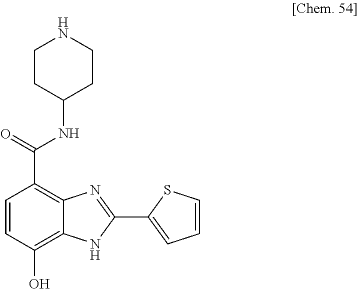

Following general procedure C, tert-Butyl 4-[7-methoxy-2-(thiophen-2-yl)-1H-benzo[d]imidazole-4-carboxamido]piperidine-1-carboxylate (0.2 g) was reacted with boron tribromide to afford the desired product (85 mg, 42% yield) as a light yellow solid: ¹H NMR (300 MHz, CD₃OD) delta 7.85-7.84 (m, 1H), 7.76 (d, J=5.1 Hz, 1H), 7.62-7.60 (m, 1H), 7.21-7.19 (m, 1H), 6.66 (d, J=5.1 Hz, 1H), 4.21-4.20 (m, 1H), 3.29-3.24 (m, 2H), 2.99-2.93 (m, 2H), 2.17-2.14 (m, 2H), 1.80-1.74 (m, 2H); ESI MS m/z 343 [C₁₇H₁₈N₄O₂S+H]⁺; HPLC >99% (AUC), t_{R}=9.07 min.

### Example 41

### 7-Hydroxy-N-(1-methylpiperidin-3-yl)-2-(thiophen-2-yl)-1H-benzo[d]imidazole-4-carboxamide

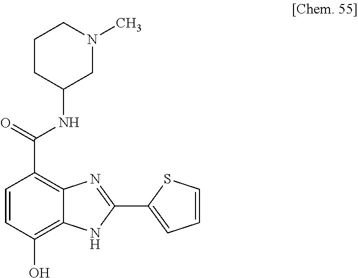

Following general procedure C, 7-methoxy-N-(1-methylpiperidin-3-yl)-2-(thiophen-2-yl)-1H-benzo[d]imidazole-4-carboxamide (0.15 g) was reacted with boron tribromide to afford the desired product (75 mg, 36% yield) as a light yellow solid: ¹H NMR (300 MHz, CD₃OD) delta 7.89-7.88 (m, 1H), 7.78 (d, J=8.4 Hz, 1H), 7.65-7.64 (m, 1H), 7.23-7.20 (m, 1H), 6.71 (d, J=8.4 Hz, 1H), 4.26-4.24 (m, 1H), 3.01-2.98 (m, 1H), 2.67-2.65 (m, 1H), 2.38 (s, 5H), 2.05-1.92 (m, 2H), 1.80-1.59 (m, 2H); ESI MS m/z 357 [C₁₈H₂₀N₄O₂S+H]⁺; HPLC 96.2% (AUC), t_{R}=9.55 min.

### Example 42

### 7-Hydroxy-N-(piperidin-4-ylmethyl)-2-(thiophen-2-yl)-1H-benzo[d]imidazole-4-carboxamide

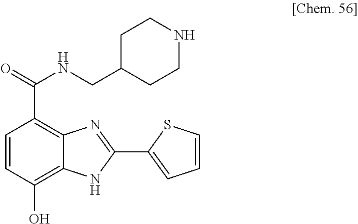

Following general procedure C, tert-Butyl 4-{[7-methoxy-2-(thiophen-2-yl)-1H-benzo[d]imidazole-4-carboxamido]methyl}piperidine-1-carboxylate (0.16 g) was reacted with boron tribromide to afford the desired product (700 mg, 35% yield) as a yellow solid: ¹H NMR (300 MHz, CD₃OD) delta 7.85-7.84 (m, 1H), 7.78-7.74 (m, 1H), 7.63-7.61 (m, 1H), 7.23-7.19 (m, 1H), 6.64-6.61 (m, 1H), 3.49 (d, J=6.6 Hz, 2H), 2.88-2.79 (m, 2H), 2.07-2.03 (m, 2H), 1.94-1.93 (m, 1H), 1.56-1.44 (m, 2H); ESI MS m/z 357 [C₁₈H₂₀N₄O₂S+H]⁺; HPLC >99% (AUC), t_{R}=9.15 min.

### Example 43

### N-(Azetidin-3-ylmethyl)-7-hydroxy-2-(thiophen-2-yl)-1H-benzo[d]imidazole-4-carboxamide

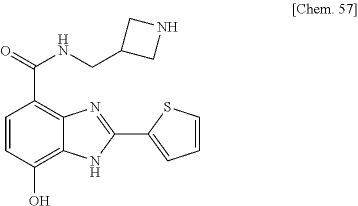

Following general procedure C, tert-Butyl 3-{[7-methoxy-2-(thiophen-2-yl)-1H-benzo[d]imidazole-4-carboxamido]methyl}azetidine-1-carboxylate (0.17 g) was reacted with boron tribromide to afford the desired product (43 mg, 24% yield) as a light yellow solid: ¹H NMR (300 MHz, CD₃OD) delta 7.84-7.83 (m, 1H), 7.74 (d, J=8.4 Hz, 1H), 7.62-7.59 (m, 1H), 7.22-7.19 (m, 1H), 6.61 (d, J=8.4 Hz, 1H), 4.02-3.96 (m, 2H), 3.90-2.84 (m, 2H), 3.74 (d, J=6.3 Hz, 2H); ESI MS m/z 329 [C₁₆H₁₆N₄O₂S+H]⁺; HPLC >99% (AUC), t_{R}=8.70 min.

### Example 44

### 7-Hydroxy-N-(pyrrolidin-3-yl)-2-(thiophen-2-yl)-1H-benzo[d]imidazole-4-carboxamide

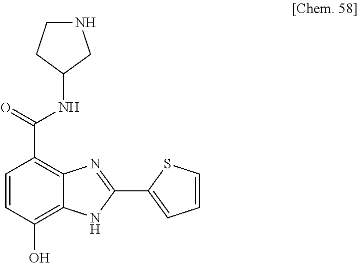

Following general procedure C, tert-Butyl 3-[7-methoxy-2-(thiophen-2-yl)-1H-benzo[d]imidazole-4-carboxamido]pyrrolidine-1-carboxylate (0.20 g) was reacted with boron tribromide to afford the desired product (0.12 g, 63% yield) as a light brown solid: ¹H NMR (300 MHz, CD₃OD) delta 8.09 (s, 1H), 7.90 (d, J=8.4 Hz, 2H), 7.36-7.33 (m, 1H), 6.87 (d, J=8.4 Hz, 1H), 4.75-4.71 (m, 1H), 3.69-3.64 (m, 2H), 3.54-3.48 (m, 2H), 2.54-2.50 (m, 1H), 2.35-2.30 (m, 1H); ESI MS m/z 329 [C₁₆H₁₆N₄O₂S+H]⁺; HPLC >99% (AUC), t_{R}=8.80 min.

### Example 45

### 7-Hydroxy-N-(piperidin-2-ylmethyl)-2-(thiophen-2-yl)-1H-benzo[d]imidazole-4-carboxamide

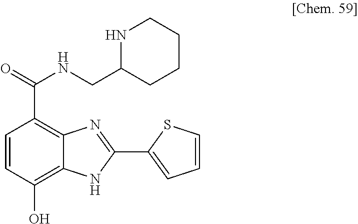

Following general procedure C, tert-Butyl 2-{[7-methoxy-2-(thiophen-2-yl)-1H-benzo[d]imidazole-4-carboxamido]methyl}piperidine-1-carboxylate (0.23 g) was reacted with boron tribromide to afford the desired product (90 mg, 44% yield) as a yellow solid: ¹H NMR (300 MHz, CD₃OD) delta 8.03-8.02 (m, 1H), 7.87 (d, J=8.4 Hz, 1H), 7.82-7.81 (m, 1H), 7.32-7.29 (m, 1H), 6.83 (d, J=8.4 Hz, 1H), 3.78-3.75 (m, 2H), 3.44-3.36 (m, 2H), 3.06-3.02 (m, 1H), 2.14-2.10 (m, 1H), 2.00-1.90 (m, 2H), 1.75-1.66 (m, 3H); ESI MS m/z 357 [C₁₈H₂₀N₄O₂S+H]⁺; HPLC >99% (AUC), t_{R}=9.49 min.

### Example 46

### 7-Hydroxy-N-(pyrrolidin-3-ylmethyl)-2-(thiophen-2-yl)-1H-benzo[d]imidazole-4-carboxamide

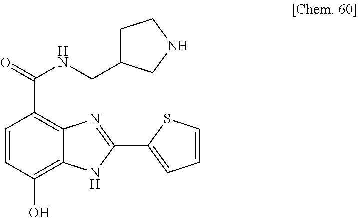

Following general procedure C, tert-Butyl 3-{[7-methoxy-2-(thiophen-2-yl)-1H-benzo[d]imidazole-4-carboxamido]methyl}pyrrolidine-1-carboxylate (0.19 g) was reacted with boron tribromide to afford the desired product (79 mg, 39% yield) as a light yellow solid: ¹H NMR (300 MHz, CD₃OD) delta 7.84-7.82 (m, 1H), 7.75 (d, J=8.4 Hz, 1H), 7.61-7.59 (m, 1H), 7.21-7.18 (m, 1H), 6.61 (d, J=8.4 Hz, 1H), 3.63-3.54 (m, 2H), 3.37-3.33 (m, 1H), 3.27-3.06 (m, 2H), 2.98-2.91 (m, 1H), 2.66-2.61 (m, 1H), 2.24-2.18 (m, 1H), 1.86-1.79 (m, 1H); ESI MS m/z 343 [C₁₇H₁₈N₄O₂S+H]⁺; HPLC >99% (AUC), t_{R}=8.91 min.

### Example 47

### N-(4-Aminocyclohexyl)-7-hydroxy-2-(thiophen-2-yl)-1H-benzo[d]imidazole-4-carboxamide

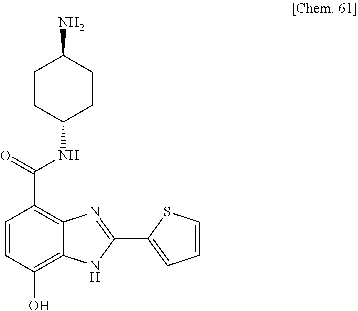

Following general procedure C, tert-Butyl-4-[7-methoxy-2-(thiophen-2-yl)-1H-benzo[d]imidazole-4-carboxamido]cyclohexylcarbamate (92 mg) was reacted with boron tribromide to afford the desired product (21 mg, 10% yield over two steps) as a light yellow solid: ¹H NMR (300 MHz, CD₃ OD) delta 7.85-7.84 (m, 1H), 7.77 (d, J=8.4 Hz, 1H), 7.61-7.59 (m, 1H), 7.22-7.17 (m, 1H), 6.63 (d, J=8.4 Hz, 1H), 4.24-4.23 (m, 1H), 3.01-2.97 (m, 1H), 2.15-2.10 (m, 2H), 2.03-1.78 (m, 6H); ESI MS m/z 357 [C₁₈H₂₀N₄O₂S+H]⁺; HPLC 95.6% (AUC), t_{R}=9.22 min.

### Example 48

### 2-(Bicyclo[2.2.1]heptan-2-yl)-7-hydroxy-N-(piperidin-3-ylmethyl)-1H-benzo[d]imidazole-4-carboxamide

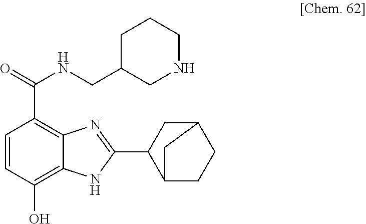

Following General Procedure C, tert-Butyl 3-((2-(bicyclo[2.2.1]heptan-2-yl)-7-methoxy-1H-benzo[d]imidazole-4-carboxamido)methyl)piperidine-1-carboxylate (330 mg crude) was reacted with boron tribromide to afford the desired product (71 mg, 45% yield) as a light yellow solid: ¹H NMR (500 MHz, CD₃OD) delta 7.75-7.68 (m, 1H), 6.58 (dd, 1H, J=4.0, 8.2 Hz), 3.47-3.36 (m, 2H), 3.27-3.20 (m, 1H), 3.11-3.05 (m, 1H), 3.01-2.96 (m, 1H, minor diastereomer), 2.69-2.62 (m, 1H), 2.57-2.51 (m 1H), 2.43-2.37 (m, 1H), 2.25-2.19 (m, 1H, minor diastereomer), 2.09-2.01 (m, 2H), 1.96-1.88 (m, 1H), 1.84-1.74 (m, 2H), 1.71-1.55 (m, 3H), 1.53-1.16 (m, 5H); ESI MS m/z 369 [C₂₁H₂₈N₄O₂+H]⁺; HPLC >99% (AUC), t_{R}=9.75 min.

### Example 49

### 2-(Bicyclo[2.2.1]heptan-2-yl)-7-hydroxy-N-(piperidin-3-yl)-1H-benzo[d]imidazole-4-carboxamide

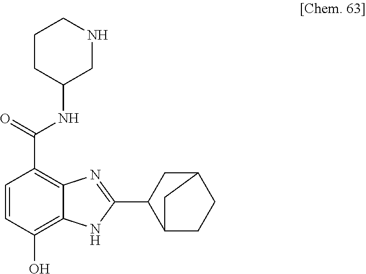

Following General Procedure C, tert-Butyl 3-(2-(bicyclo[2.2.1]heptan-2-yl)-7-methoxy-1H-benzo[d]imidazole-4-carboxamido)piperidine-1-carboxylate (210 mg crude) was reacted with boron tribromide to afford the desired product (72 mg, 43% yield) as a light yellow solid: ¹H NMR (300 MHz, CD₃ OD) delta 7.69 (dd, J=3.6, 8.1 Hz, 1H), 6.61 (dd, J=2.7, 8.1 Hz, 1H), 4.12-4.01 (m, 1H), 3.45-3.36 (m, 1H), 3.03-2.93 (m, 1H), 2.78-2.52 (m, 3H), 2.44-2.36 (m, 1H), 2.25-1.16 (m 13H); ESI MS m/z 355 [C₂₀H₂₆N₄O₂+H]⁺; HPLC >99% (AUC), t_{R}=9.55 min (minor diastereomer), 9.74 min (major diastereomer).

General Procedure D—Synthesis of Compounds of Formula I-II as Described in Scheme (1):

To a solution of acid (1.0 equiv) in DMF (5-10 mL) was added HATU (1.2-1.5 equiv), DIPEA (3.0-5.0 equiv), and the amine (1.5-2.0 equiv) and the reaction mixture was either stirred at room temperature for 16 h or heated at 50-70 degrees C. for 16 h. The reaction mixture was diluted with satd. aq NaHCO₃ (20 mL) and extracted with ethyl acetate (3×20 mL). The combined organic layer was dried over Na₂SO₄, concentrated, and purified by preparative HPLC (C18 silica, 10-90% acetonitrile/water with 0.05% TFA). The desired product was obtained as the trifluoroacetic acid salt which was eluted through an ion-exchange column (using methanol and 7 N methanol in ammonia) to obtain the desired products. In some instances the desired product was treated with TFA (1-2 mL) for 1 h, concentrated and purified by preparative HPLC (C18 silica, 10-90% acetonitrile/water with 0.05% TFA). The desired product was obtained as the trifluoroacetic acid salt which was eluted through an ion-exchange column (using methanol and 7 N methanol in ammonia) to obtain the desired products

### Example 50

### (S)-tert-Butyl 3-(2-(5-bromothiophen-2-yl)-7-hydroxy-1H-benzo[d]imidazole-4-carboxamido)piperidine-1-carboxylate

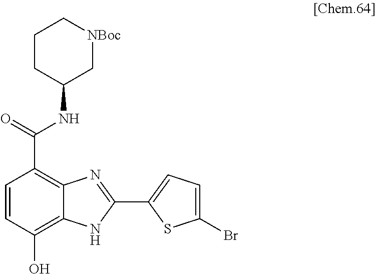

Following General Procedure D, 2-(5-bromothiophen-2-yl)-7-hydroxy-1H-benzo[d]imidazole-4-carboxylic acid (90 mg, 0.27 mmol) was reacted with (S)-tert-butyl 3-aminopiperidine-1-carboxylate (106 mg, 0.53 mmol) to afford the desired product (48 mg, 35% yield) as yellow-brown solid: ¹H NMR (500 MHz, CD₃OD) delta 7.84 (d, J=8.5 Hz, 1H), 7.71 (s, 1H), 7.28 (s, 1H), 6.78 (d, J=8.5 Hz, 1H), 4.21 (bs, 1H), 3.86 (bs, 1H), 3.58-3.18 m, 2H), 2.14-2.03 (m, 2H), 1.89 (bs, 1H), 1.59 (bs, 1H), 1.17 (bs, 1H); ESI MS m/z 521 [C₂₂H₂₅BrN₄O₄S]⁺; HPLC >99% (AUC), t_{R}=15.30 min.

### Example 51

### (S)-2-(5-bromothiophen-2-yl)-7-hydroxy-N-(piperidin-3-yl)-1H-benzo[d]imidazole-4-carboxamide

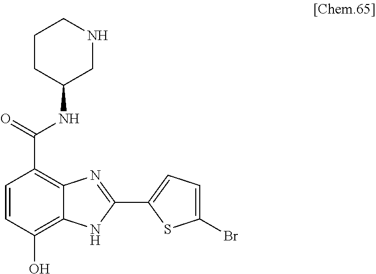

A solution of (S)-tert-butyl 3-(2-(5-bromothiophen-2-yl)-7-hydroxy-1H-benzo[d]imidazole-4-carboxamido)piperidine-1-carboxylate (35 mg, 0.067 mmol) in CH₂Cl₂ (1 mL) and TFA (1 mL) was stirred at room temperature for 1 h. The reaction mixture was concentrated and purified by purified by preparative HPLC (C18 silica, 10-90% acetonitrile/water with 0.05% TFA). The desired product was obtained as the trifluoroacetic acid salt which was eluted through an ion-exchange column (using methanol and 7 N methanol in ammonia) to obtain the desired (20 mg, 72%) as yellow solid: ¹H NMR (500 MHz, DMSO-d₆) delta 13.61 (s, 1H), 11.00 (s, 1H), 9.57 (d, J=6.5 Hz, 1H), 8.75 (bs, 1H), 7.89 (d, J=4.0 Hz, 1H), 7.72 (d, J=8.0 Hz, 1H), 7.41 (d, J=3.5 Hz, 1H), 6.77 (d, J=8.5 Hz, 1H), 3.46 (d, J=8.5 Hz, 1H), 3.21 (d, J=12.5 Hz, 1H), 3.04-2.96 (m, 2H), 2.10 (bs, 1H), 2.03-2.00 (m, 2H), 1.85-1.70 (m, 4H), 0.68 (bs, 1H); ESI MS m/z 421 [C₁₇H₁₇BrN₄O₂S]⁺; HPLC 98.34% (AUC), t_{R}=8.17 min.

### Example 52

### 2-(Bicyclo[2.2.1]heptan-2-yl)-7-hydroxy-N-((S)-piperidin-3-yl)-1H-benzo[d]imidazole-4-carboxamide

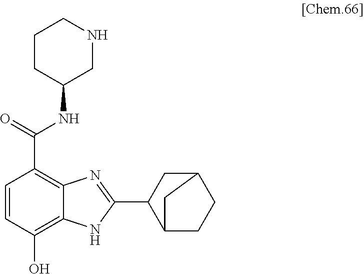

Following General Procedure C, (3S)-tert-Butyl 3-(2-(bicyclo[2.2.1]heptan-2-yl)-7-methoxy-1H-benzo[d]imidazole-4-carboxamido)piperidine-1-carboxylate (230 mg crude) was reacted with boron tribromide to afford the desired product (103 mg, 52% over two steps) as a light brown solid: ¹H NMR (300 MHz, CD₃OD) delta 7.69 (dd, J=3.6, 8.4 Hz, 1H), 6.60 (dd, J=2.7, 8.4 Hz, 1H), 4.12-4.02 (m, 1H), 3.46-3.35 (m, 1H), 3.03-2.93 (m, 1H), 2.78-2.60 (m, 3H), 2.56-2.36 (m, 1H), 2.25-1.17 (m, 13H); ESI MS m/z 355 [C₂₀H₂₆N₄O₂+H]⁺; HPLC 99.0% (AUC), t_{R}=9.35 min (minor diastereomer), 9.49 min (major diastereomer).

### Example 53

### 2-(Bicyclo[2.2.1]heptan-2-yl)-7-hydroxy-N-(adamantane-3-ylamino)-1H-benzo[d]imidazole-4-carboxamide

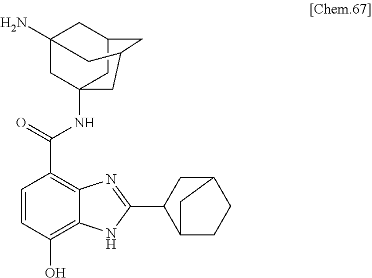

Following General Procedure C, tert-Butyl 3-{[2-(bicyclo[2.2.1]heptan-2-yl)-7-methoxy-1H-benzo[d]imidazole-4-carboxamido]methyl}adamantane-1-carboxylate (140 mg crude) was reacted with boron tribromide to afford the desired product (57 mg, 31% over two steps) as a light yellow solid: ¹H NMR (300 MHz, CD₃OD) delta 7.66-7.62 (m, 1H), 6.57-6.53 (m, 1H), 3.45-3.35 (m, 1H), 3.00-2.90 (m, 1H, minor diastereomer), 2.68-2.62 (m, 1H, major diastereomer), 2.56-2.52 (m, 1H, minor diastereomer), 2.43-2.18 (m, 7H), 2.13-1.99 (m 3H), 1.84-1.21 (m, 12H); ESI MS m/z 421 [C₂₅H₃₂N₄O₂+H]⁺; HPLC 96.6% (AUC), t_{R}=10.45 min.

### Example 54

### 2-(Thiophene-2-yl)-7-hydroxy-N-(adamantate-3-ylamino)-1H-benzo[d]imidazole-4-carboxamide

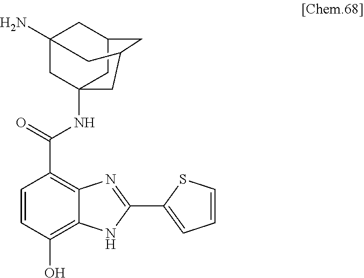

Following General Procedure C, tert-Butyl 3-((2-thiophene-2-yl)-7-methoxy-1H-benzo[d]imidazole-4-carboxamido)methyl)adamantane-1-carboxylate (110 mg) was reacted with boron tribromide to afford the desired product (62 mg, 28% over two steps) as a light yellow solid: ¹H NMR (300 MHz, CD₃ OD) delta 7.80 (d, J=3.9 Hz, 1H), 7.69 (d, J=8.4 Hz, 1H), 7.58 (d, 4.8 Hz), 7.20-7.17 (m, 1H), 6.59 (d, 1H, J=8.4 Hz), 2.38-2.11 (m, 8H), 1.86-1.63 (m, 6H); ESI MS m/z 409 [C₂₂H₂₄N₄O₂S+H]⁺; HPLC >99% (AUC), t_{R}=11.27 min.

### Example 55

### N-(3-Aminocyclohexyl)-2-(bicyclo[2.2.1]heptan-2-yl)-7-hydroxy-1H-benzo[d]imidazole-4-carboxamide

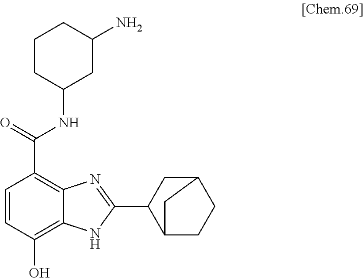

Following General Procedure C, tert-Butyl 3-(2-(bicyclo[2.2.1]heptan-2-yl)-7-methoxy-1H-benzo[d]imidazole-4-carboxamido)cyclohexylcarbamate (120 mg crude) was reacted with boron tribromide to afford the desired product (66 mg, 40% yield) as a light yellow solid: ¹H NMR (300 MHz, CD₃ OD) delta 7.72-7.67 (m, 1H), 6.58-6.55 (m, 1H), 4.57-4.48 (m, 1H, minor diastereomer), 4.03-3.90 (m, 1H, major diastereomer), 3.45-3.35 (m, 1H), 3.03-2.90 (m, 1H), 2.66-2.52 (m, 1H), 2.44-2.32 (m 2H, major diastereomer), 2.22-1.14 (m, 15H); ESI MS m/z 369 [C₂₁H₂₈N₄O₂+H]⁺; HPLC >99% (AUC), t_{R}=9.40, 9.53, 9.58, 9.81 min (mixture of diastereomers).

### Example 56

### N-{[(cis)-4-Aminocyclohexyl]methyl}-2-(bicyclo[2.2.1]heptan-2-yl)-7-hydroxy-1H-benzo[d]imidazole-4-carboxamide

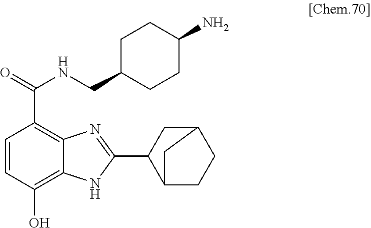

Following General Procedure C, tert-Butyl (cis)-4-{[2-(bicyclo[2.2.1]heptan-2-yl)-7-methoxy-1H-benzo[d]imidazole-4-carboxamido]methyl}cyclohexylcarbamate (220 mg crude) was reacted with boron tribromide to afford the desired product (64 mg, 53% over two steps) as a light yellow solid: ¹H NMR (300 MHz, CD₃OD) delta 7.69 (dd, J=3.9, 8.4 Hz, 1H), 6.59-6.54 (m, 1H), 3.56-3.37 (m, 2H), 3.15-3.07 (m, 1H), 3.00-2.90 (m, 1H, minor diastereomer), 2.74-2.66 (m, 1H, minor diastereomer), 2.55-2.51 (m, 1H, minor diastereomer), 2.42-2.34 (m, 1H), 2.25-2.16 (m, 1H, minor diastereomer), 2.06-1.98 (m, 1H), 1.80-1.20 (m, 14H); ESI MS m/z 383 [C₂₂H₃₀N₄O₂+H]⁺; HPLC 99.0% (AUC), t_{R}=9.53, 9.88, 9.96 min (mixture of diastereomers).

### Example 57

### (S)-7-hydroxy-2-(5-(piperazin-1-yl)thiophen-2-yl)-N-(piperidin-3-yl)-1H-benzo[d]imidazole-4-carboxamide

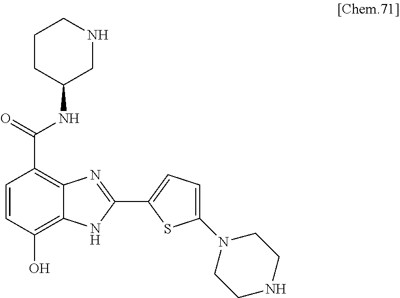

A mixture of (S)-tert-Butyl 3-(2-(5-bromothiophen-2-yl)-7-hydroxy-1H-benzo[d]imidazole-4-carboxamido)piperidine-1-carboxylate (0.12 g, 0.24 mmol), tert-butyl piperazine-1-carboxylate (110 mg, 0.60 mmol), CuI (5.7 mg, 0.030 mmol), Cu (2.0 mg, 0.030 mmol), K₃PO₄ H₂O (160 mg, 0.72 mmol) in 2-(dimethylamino)ethanol (2 mL) was stirred at 75 degrees C. for 18 h. The reaction mixture was cooled, concentrated, dissolved in CH₃OH (3 mL) and filtered. The filtrate was purified by preparative HPLC (C18 silica, 10-90% acetonitrile/water with 0.05% TFA). The desired fractions were combined, concentrated and the residue was dissolved in CH₂Cl₂ (2 mL) and TFA (1 mL) and stirred at rt for 30 min. The reaction mixture was concentrated and the residue was eluted through an ion-exchange column (SCX-2) (using methanol and 7 N methanol in ammonia) to obtain the desired product (7 mg, 14% yield) as a yellow solid: ¹H NMR (500 MHz, CD₃OD) delta 8.20 (d, J=4.5 Hz, 1H), 7.50 (d, J=4.5 Hz, 1H), 7.14 (d, J=4.0 Hz, 1H), 6.54 (d, J=3.5 Hz, 1H), 4.20-4.16 (m, 1H), 3.43 (dd, J=12.5, 3.5 Hz, 1H), 3.19-3.15 (m, 2H), 3.10-2.97 (m, 3H), 2.05-1.96 (m, 2H), 1.84-1.72 (m, 3H), 1.19-1.16 (m, 1H), 1.13-1.08 (m, 1H); ESI MS m/z 427 [C₂₁H₂₆N₆O₂S+H]⁺ HPLC 97.13% (AUC), t_{R}=8.29 min.

### Example 58

### (R)-7-hydroxy-N-(piperidin-3-ylmethyl)-2-(thiophen-2-ylmethyl)-1H-benzo[d]imidazole-4-carboxamide

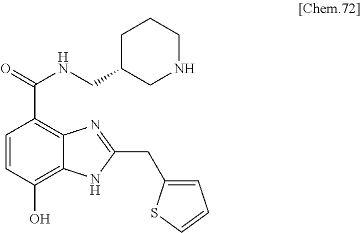

Following General Procedure D, 2-(5-bromothiophen-2-yl)-7-hydroxy-1H-benzo[d]imidazole-4-carboxylic acid (0.13 mg, 0.47 mmol) was reacted with (S)-tert-butyl 3-(aminomethyl)piperidine-1-carboxylate (200 mg, 0.93 mmol) and the intermediate was treated with TFA to afford the desired product (15 mg, 31% yield) as yellow solid: ¹H NMR (500 MHz, CD₃OD) delta 7.75 (d, J=8.5 Hz, 1H), 7.30 (dd, J=5.5, 1.5 Hz, 1H), 7.02-7.01 (m, 1H), 6.98 (dd, J=5.0, 3.5 Hz, 1H), 6.70 (d, J=8.5 Hz, 1H), 4.52 (s, 2H), 3.53-3.45 (m, 2H), 3.37 (dd, J=9.0, 6.0 Hz, 1H), 2.95-2.89 (m, 2H), 2.82 (t, J=12.0 Hz, 1H), 2.15-2.11 (m, 1H), 2.00-1.94 (m, 3H), 1.78-1.74 (m, 1H), 1.44-1.36 (m, 2H); ESI MS m/z 371 [C₁₉H₂₂N₄O₂S+H]⁺ HPLC 95.5% (AUC), t_{R}=7.17 min.

### Example 59

### (S)-7-hydroxy-N-(piperidin-3-yl)-2-(thiophen-2-ylmethyl)-1H-benzo[d]imidazole-4-carboxamide

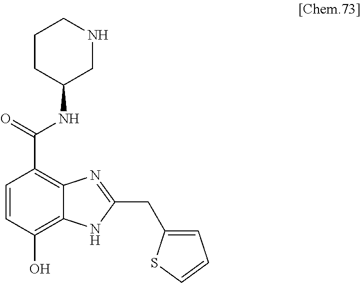

Following General Procedure D, 2-(5-bromothiophen-2-yl)-7-hydroxy-1H-benzo[d]imidazole-4-carboxylic acid (0.17 mg, 0.62 mmol) was reacted with (5)-tert-butyl 3-aminopiperidine-1-carboxylate (250 mg, 1.3 mmol) and the intermediate was treated with TFA to afford the desired product (25 mg, 68% yield) as yellow solid: ¹H NMR (500 MHz, CD₃OD) delta 7.63 (d, J=8.5 Hz, 1H), 7.20 (dd, J=5.0, 1.0 Hz, 1H), 6.91 (dd, J=5.0, 3.5 Hz, 1H), 6.57 (d, J=8.5 Hz, 1H), 4.37 (s, 2H), 4.15-4.11 (m, 1H), 3.37 (dd, J=10.5, 3.5 Hz, 1H), 3.14-3.11 (m, 1H), 2.93-2.86 (m, 2H), 2.04-2.01 (m, 1H), 1.94-1.91 (m, 1H), 1.74-1.65 (m, 2H).; ESI MS m/z 357 [C₁₈H₂₀N₄O₂S+H]⁺ HPLC 96.59% (AUC), t_{R}=7.07 min.

### Example 60

### (S)-7-hydroxy-N-(piperidin-3-ylmethyl)-2-(thiophen-2-ylmethyl)-1H-benzo[d]imidazole-4-carboxamide

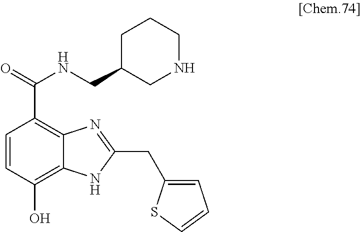

Following General Procedure D, 2-(5-bromothiophen-2-yl)-7-hydroxy-1H-benzo[d]imidazole-4-carboxylic acid (0.13 mg, 0.47 mmol) was reacted with (R)-tert-butyl 3-(aminomethyl)piperidine-1-carboxylate (200 mg, 0.93 mmol) and the intermediate was treated with TFA to afford the desired product (12 mg, 28% yield) as yellow solid: ¹H NMR (500 MHz, CD₃OD) delta 7.75 (d, J=8.5 Hz, 1H), 7.30 (dd, J=5.5, 1.5 Hz, 1H), 7.02-7.01 (m, 1H), 6.98 (dd, J=5.0, 3.5 Hz, 1H), 6.70 (d, J=5.0 Hz, 1H), 4.50 (s, 2H), 3.51-3.48 (m, 2H), 3.37 (dd, J=13.0, 7.0 Hz, 1H), 2.92-2.89 (m, 2H), 2.80 (t, J=12.0 Hz, 1H), 2.16-2.10 (m, 1H), 2.00-1.95 (m, 3H), 1.80-1.72 (m,1H), 1.44-1.39 (m, 2H); ESI MS m/z 371 [C₁₉H₂₂N₄O₂S+H]⁺ HPLC 96.8% (AUC), t_{R}=6.93 min.

### Example 61

### Step 1: Synthesis of Methyl 3-(5-bromothiophene-2-carboximidamido)-4-methoxybenzoate Hydrochloride

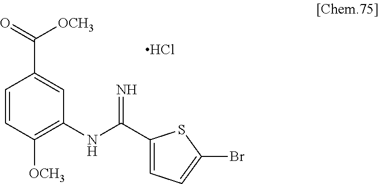

Following the procedure outlined for step 1 in Example 1, methyl 3-amino-4-methoxybenzoate (1.5 g, 7.9 mmol) was reacted with 5-bromothiophene-2-carbonitrile (3.0 g, 16 mmol) to afford the desired product (1.6 g, 54% yield) as a dark brown solid: ESI MS m/z 368 [C₁₄H₁₃BrN₂O₃S+H]⁺.

### Step 2: Synthesis of Methyl 2-(5-bromothiophen-2-yl)-7-methoxy-1H-benzo[d]imidazole-4-carboxylate

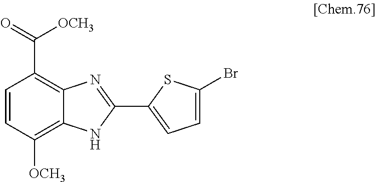

Following the procedure outlined for step 2 in Example 1, methyl 3-(5-bromothiophene-2-carboximidamido)-4-methoxybenzoate hydrochloride (1.7 g, 4.2 mmol) was reacted with 5% aq NaOCl and satd. aq NaHCO₃ to afford the desired product (0.45 g, 30% yield) as a brown solid: ESI MS m/z 369 [C₁₄H₁₁BrN₂O₃S+H]⁺.

### Step 3: Synthesis of 2-(5-Bromothiophen-2-yl)-7-hydroxy-1H-benzo[d]imidazole-4-carboxylic acid

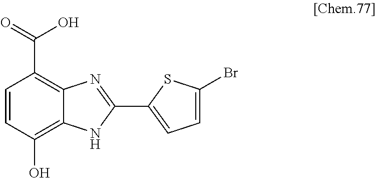

Following the procedure outlined for step 4 in Example 1, methyl 2-(5-bromothiophen-2-yl)-7-methoxy-1H-benzo[d]imidazole-4-carboxylate (0.40 g, 1.1 mmol) was reacted with boron tribromide (1.5 g, 6.6 mmol) to afford the desired product (0.34 g, 92% yield) as a light brown solid: ESI MS m/z 340 [C₁₂H₇BrN₂O₃S+H]⁺.

### Example 62

### Step 1: Synthesis of Methyl 4-methoxy-3-(2-(thiophen-2-yl)acetimidamido)benzoate Hydrochloride

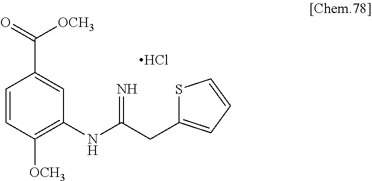

Following the procedure outlined for step 1 in Example 1, methyl 3-amino-4-methoxybenzoate (2.2 g, 12 mmol) was reacted with 2-(thiophen-2-yl)acetonitrile (3.0 g, 24 mmol) to afford the desired product (3.2 g, 78% yield) as a yellow brown solid: ESI MS m/z 305 [C₁₅H₁₆N₂O₃S+H]⁺.

### Step 2: Synthesis of Methyl 7-methoxy-2-(thiophen-2-ylmethyl)-1H-benzo[d]imidazole-4-carboxylate

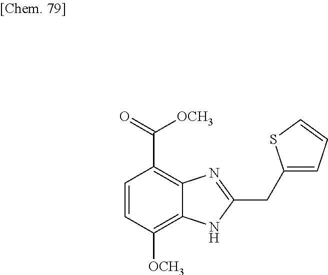

Following the procedure outlined for step 2 in Example 1, methyl 4-methoxy-3-(2-(thiophen-2-yl)acetimidamido)benzoate hydrochloride (3.1 g, 10 mmol) was reacted with 5% aq NaOCl and satd. aq NaHCO₃ to afford the desired product (1.1 g, 30% yield) as a brown solid: ESI MS m/z 303 [C₁₅H₁₄N₂O₃S+H]⁺.

### Step 3: Synthesis of 7-hydroxy-2-(thiophen-2-ylmethyl)-1H-benzo[d]imidazole-4-carboxylic acid

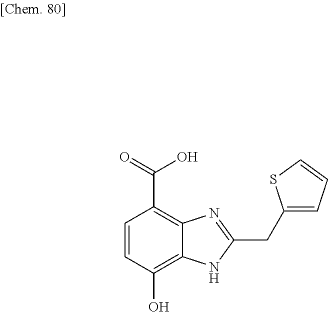

Following the procedure outlined for step 4 in Example 1, methyl 7-methoxy-2-(thiophene-2-ylmethyl)-1H-benzo[d]imidazole-4-carboxylate (0.91 g, 3.0 mmol) was reacted with boron tribromide (4.5 g, 18 mmol) to afford the desired product (0.63 g, 73% yield) as a light brown solid: ESI MS m/z 275 [C₁₃H₁₀N₂O₃S+H]⁺.

### Example 63

### Step 1: Synthesis of Methyl 3-(bicyclo[2.2.1]heptane-2-carboximidamido)-4-methoxybenzoate

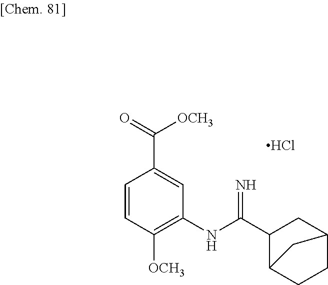

Following the procedure outlined for step 1 in Example 1, methyl-3-amino-4-methoxy benzoate (7.5 g, 41 mmol) was reacted with 2-norbornane carbonitrile (10 g, 82 mmol) to afford product (11 g, 90%) as a white solid: ¹H NMR (300 MHz, DMSO-d₆) delta 8.29-8.20 (m, 1H), 7.99-7.96 (m, 1H), 7.33-7.28 (m, 1H), 3.88 (s, 3H), 3.84 (s, 3H), 2.70-2.62 (m, 1H), 1.87-1.17 (m, 8H); ESI MS m/z 303 [C₁₇H₂₂N₂ O₃+H]⁺.

### Step 2: Synthesis of Methyl 2-(bicyclo[2.2.1]heptan-2-yl)-7-methoxy-1H-benzo[d]imidazole-4-carboxylate

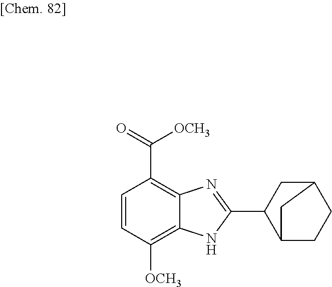

Following the procedure outlined for step 2 in Example 1, methyl 3-(bicyclo[2.2.1]heptane-2-carboximidamido)-4-methoxybenzoate (11 g, 37 mmol) was reacted with NaOCl (33 mL, 10-13%, 44 mmol) and chromatographed (hexanes/ethyl acetate) to afford product (3.9 g, 36%) as a foam: ¹H NMR (300 MHz, DMSO-d₆) delta 12.05 (s, 1H, tautomer 1), 11.97 (s, 1H, tautomer 2), 7.73 (dd, 1H, J=1.2, 8.7 Hz), 6.78 (dd, 1H, J=2.4, 8.7 Hz), 4.00 (s, 3H, tautomer 1), 3.98 (s, 3H, tautomer 2), 3.90 (s, 3H, tautomer 1), 3.89 (s, 3H, tautomer 2), 3.47-3.41 (m, 1H, tautomer 1), 3.11-3.06 (m, 1H, tautomer 2), 2.70-2.66 (m, 1H, tautomer 1), 2.38-2.18 (m, 2H), 2.08-2.00 (m, 1H, tautomer 1), 1.91-1.80 (m, 1H, tautomer 2), 1.68-1.24 (m, 5H), 1.11-0.98 (m, 1H); ESI MS m/z 301 [C₁₇H₂₀N₂O₃+H]⁺.

### Step 3: Synthesis of 2-(Bicyclo[2.2.1]heptan-2-yl)-7-methoxy-1H-benzo[d]imidazole-4-carboxylic Acid

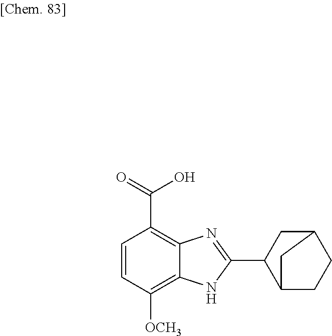

Following the procedure outlined for step 3 in Example 1, methyl 2-(bicyclo[2.2.1]heptan-2-yl)-7-methoxy-1H-benzo[d]imidazole-4-carboxylate (3.9 g, 13 mmol) was reacted with sodium hydroxide (30 mL, 3 M) to afford crude product (3.6 g) as a white solid: ESI MS m/z 287 [C₁₆H₁₈N₂O₃+H]⁺.

### Example 64

### tert-Butyl 3-{[2-(bicyclo[2.2.1]heptan-2-yl)-7-methoxy-1H-benzo[d]imidazole-4-carboxamido]methyl}piperidine-1-carboxylate

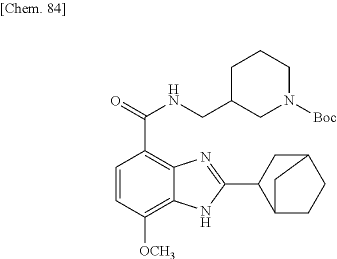

Following General Procedure D 2-(bicyclo[2.2.1]heptan-2-yl)-7-methoxy-1H-benzo[d]imidazole-4-carboxylic acid (125 mg, 0.43 mmol) was reacted with tert-butyl 3-(aminomethyl)piperidine-1-carboxylate (138 mg, 0.65 mmol) to afford the desire product (338 mg crude) as an oil: ESI MS m/z 483 [C₂₇H₃₈N₄O₄+H]⁺.

### Example 65

### tert-Butyl 3-((2-(bicyclo[2.2.1]heptan-2-yl)-7-methoxy-1H-benzo[d]imidazole-4-carboxamido)methyl)adamantane-1-carboxylate

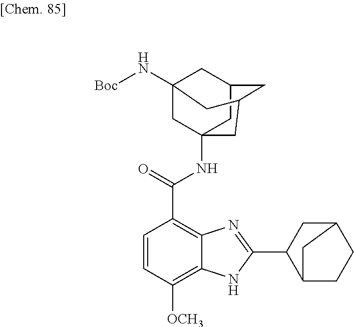

Following General Procedure D 2-(bicyclo[2.2.1]heptan-2-yl)-7-methoxy-1H-benzo[d]imidazole-4-carboxylic acid (125 mg, 0.43 mmol) was reacted with tert-butyl 3-aminoadamantanecarboxylate (176 mg, 0.65 mmol) to afford the desire product (145 mg crude) as an oil: ESI MS m/z 535 [C₃₁H₄₂N₄O₄+H]⁺.

### Example 66

### (3S)-tert-Butyl 3-(2-(bicyclo[2.2.1]heptan-2-yl)-7-methoxy-1H-benzo[d]imidazole-4-carboxamido)piperidine-1-carboxylate

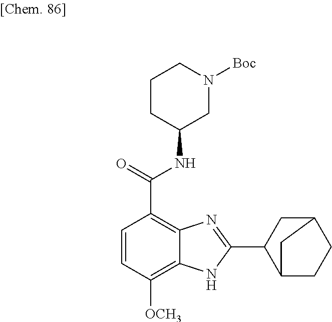

Following General Procedure D 2-(bicyclo[2.2.1]heptan-2-yl)-7-methoxy-1H-benzo[d]imidazole-4-carboxylic acid (150 mg, 0.54 mmol) was reacted with (S)-tert-butyl 3-aminopiperidine-1-carboxylate (160 mg, 0.81 mmol) to afford the desire product (237 mg crude) as an oil: ESI MS m/z 467 [C₂₆H₃₆N₄O₄+H]⁺.

### Example 67

### tert-Butyl (cis)-4-((2-(bicyclo[2.2.1]heptan-2-yl)-7-methoxy-1H-benzo[d]imidazole-4-carboxamido)methyl)cyclohexylcarbamate

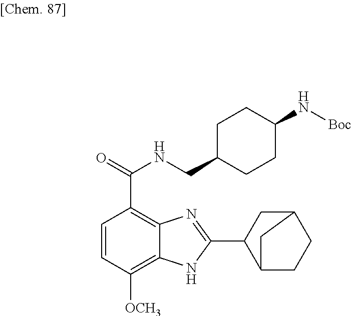

Following General Procedure D 2-(bicyclo[2.2.1]heptan-2-yl)-7-methoxy-1H-benzo[d]imidazole-4-carboxylic acid (90 mg, 0.31 mmol) was reacted with tert-butyl (1s,4s)-4-(aminomethyl)cyclohexylcarbamate (71 mg, 0.31 mmol) to afford the desire product (237 mg crude) as an oil: ESI MS m/z 497 [C₂₈H₄₀N₄O₄+H]⁺.

### Example 68

### tert-Butyl 3-((2-thiophene-2-yl)-7-methoxy-1H-benzo[d]imidazole-4-carboxamido)methyl)adamantane-1-carboxylate

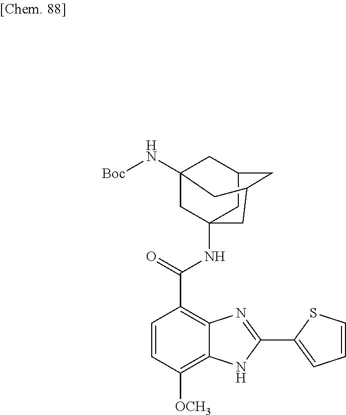

Following General Procedure B, 7-methoxy-2-(thiophen-2-yl)-1H-benzo[d]imidazole-4-carboxylic acid (0.15 g, 0.55 mmol) was reacted with tert-butyl 3-aminoadamantanecarboxylate (0.22 g, 0.82 mmol) to afford the desired product (118 mg crude) as a white solid: ESI MS m/z 523 [C₂₈H₃₄ N₄O₄S+H]⁺.

### Example 69

### tert-Butyl 3-(2-(bicyclo[2.2.1]heptan-2-yl)-7-methoxy-1H-benzo[d]imidazole-4-carboxamido)piperidine-1-carboxylate

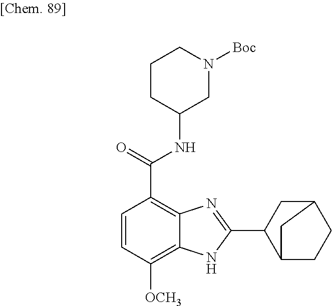

Following General Procedure B, 2-(Bicyclo[2.2.1]heptan-2-yl)-7-methoxy-1H-benzo[d]imidazole-4-carboxylic acid (150 mg, 0.55 mmol) was reacted with tert-butyl 3-aminopiperidine-1-carboxylate (0.22 g, 1.1 mmol) to afford the desired product (219 mg crude) as a foam: ESI MS m/z 469 [C₂₆H₃₆N₄O₄+H]⁺.

### Example 70

### tert-Butyl 3-(2-(bicyclo[2.2.1]heptan-2-yl)-7-methoxy-1H-benzo[d]imidazole-4-carboxamido)cyclohexylcarbamate

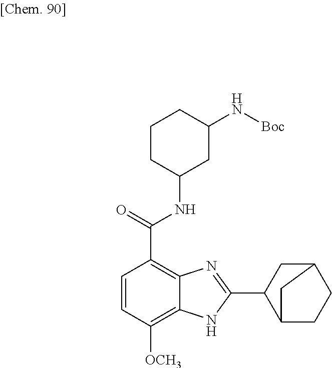

Following General Procedure B, 2-(Bicyclo[2.2.1]heptan-2-yl)-7-methoxy-1H-benzo[d]imidazole-4-carboxylic acid (150 mg, 0.55 mmol) was reacted with tert-butyl 3-aminocyclohexylcarbamate (0.24 g, 1.1 mmol) to afford the desired product (126 mg crude) as a glass: ESI MS m/z 483 [C₂₇H₃₈N₄O₄+H]⁺.

### Example 71

### 4-[7-methoxy-2-(thiophen-2-yl)-1H-benzo[d]imidazole-4-carboxamido]cyclohexane carboxylic Acid

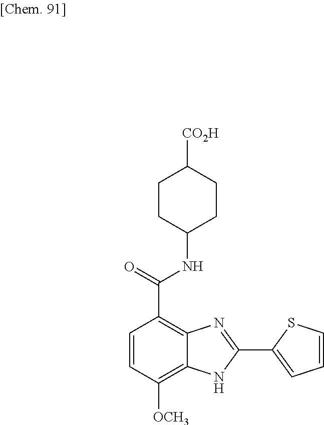

Following General Procedure B, 7-methoxy-2-(thiophen-2-yl)-1H-benzo[d]imidazole-4-carboxylic acid (140 mg, 0.52 mmol) was reacted with 4-aminocyclohexanecarboxylic acid (150 mg, 1.0 mmol) to afford the desired product (53 mg) as a yellow solid: ESI MS m/z 400 [C₂₀H₂₁N₃O₄S+H]⁺.

### Example 72

### 4-[7-hydroxy-2-(thiophen-2-yl)-1H-benzo[d]imidazole-4-carboxamido]cyclohexanecarboxylic Acid

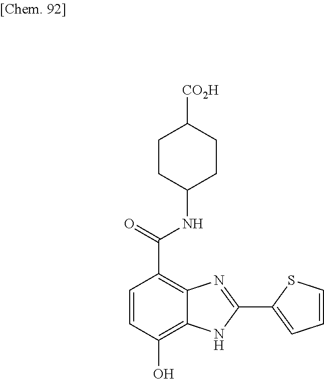

Following General Procedure C, 4-[7-methoxy-2-(thiophen-2-yl)-1H-benzo[d]imidazole-4-carboxamido]cyclohexanecarboxylic acid (53 mg) was reacted with boron tribromide to afford the desired product (18 mg, 35% yield) as a light green solid: ¹H NMR (300 MHz, CD₃OD) delta 8.04 (d, J=3.6 Hz, 1H, minor diastereomer), 7.97 (d, J=3.6 Hz, major diastereomer), 7.81 (d, J=8.4 Hz, 1H), 7.72 (d, J=4.8 Hz, 1H), 7.33-7.30 (m, 1H, minor diastereomer), 7.25 (dd, J=4.8, 3.6 Hz, 1H, major diastereomer), 6.82 (d, J=8.4 Hz, 1H, minor diastereomer), 6.77 (d, J=8.4 Hz, 1H, major diastereomer), 4.32-4.26 (m, 1H), 2.58-2.49 (m, 1H), 2.20-1.82 (m, 8H); ESI MS m/z 386 [C₁₉H₁₉N₃O₄S+H]⁺; HPLC 97.3% (AUC), t_{R}=12.07 mM (minor diastereomer), 12.37 mM (major diastereomer).

### Examples 73

Kinase Assay

GSK-3beta activity was measured in the presence or absence of compounds using Z′-LYTE kinase assay (Rodems S M, et al., Assay Drug Dev Technol. 1: 9-19, 2002.) kit with SER/THR 9 peptide (Invitrogen) following the manufacturer's instruction. The Z′-LYTE kinase assay kit employs a fluorescence resonance energy transfer (FRET) between two fluorophores, coumarin and fluorescein, attached to each end of a substrate peptide.

Test compounds were dissolved in DMSO at 12.5 mM and then serially diluted as the DMSO concentration in the assays to be 1%. The serially diluted compounds, 0.04 ng/micro-1 GSK-3beta (Invitrogen) and 2 micro-M SER/THR 9 peptide were reacted in a reaction buffer (50 mM HEPES pH 7.5, 0.01% Brij-35, 10 mM MgCl₂, 1 mM EGTA, 15 micro-M ATP). For 0% phosphorylation control, ATP was omitted from the reaction mixture. For 100% phosphorylation control, SER/THR 9 phosphopeptide was used in place of the SER/THR 9 peptide. Following 1 hour incubation at room temperature, the reaction was stopped by the addition of half assay volume of development solution and further incubated for 1 hour at room temperature. After adding the half assay volume of stop reagent, emission signals of coumarin and fluorescein were measured by Wallac EnVision 2103 multilabel reader (PerkinElmer). The extent of phosphorylation was determined according to the 0% and 100% phosphorylation control samples using the following equation:

$$\% phosphorylation = 1 - \frac{( emission ratio \times {F}_{100 \%} ) - {C}_{100 \%}}{( {C}_{0 \%} - {C}_{100 \%} ) + [ emission ratio \times ( {F}_{100 \%} - {F}_{0 \%} ) ]} \\ where \text{:} emission ratio = \frac{coumarin emission signal ( 445 nm )}{fluorescein emission signal ( 520 nm )} \\ {C}_{100 \%} = coumarin emission signal of the 100 \% phosphorylation control {C}_{0 \%} = coumarin emission signal of the 0 \% phosphorylation control \\ {F}_{100 \%} = fluorescein emission signal of the 100 \% phosphorylation control {F}_{0 \%} = fluorescein emission signal of the 0 \% phosphorylation control \qquad [ Math . 1 ]$$

IC₅₀ values were calculated by nonlinear four parameter fit using SigmaPlot, version 10.0 (Systat Software, Inc.).

IC₅₀ values of the compounds in the present invention are shown in following table 2:

TABLE 2

| Example No. | compound | IC₅₀ (micro-M) |
|---|---|---|
| 39 | 7-Hydroxy-N-(piperidin-3-ylmethyl)-2-(thiophen-2-yl)-1H- | 0.0086 |
| | benzo[d]imidazole-4-carboxamide | |
| 38 | (S)-7-Hydroxy-N-(piperidin-3-yl)-2-(thiophen-2-yl)-1H- | 0.01 |
| | benzo[d]imidazole-4-carboxamide | |
| 35 | 7-Hydroxy-N-(piperidin-3-yl)-2-(thiophen-2-yl)-1H- | 0.017 |
| | benzo[d]imidazole-4-carboxamide | |
| 40 | 7-Hydroxy-N-(piperidin-4-yl)-2-(thiophen-2-yl)-1H- | 0.018 |
| | benzo[d]imidazole-4-carboxamide | |
| 46 | 7-Hydroxy-N-(pyrrolidin-3-ylmethyl)-2-(thiophen-2-yl)- | 0.025 |
| | 1H-benzo[d]imidazole-4-carboxamide | |
| 20 | 7-Hydroxy-N-(4-hydroxycyclohexyl)-2-(thiophen-2-yl)- | 0.026 |
| | 1H-benzo[d]imidazole-4-carboxamide | |
| 10 | 2-cyclopropyl-4-hydroxy-N-(piperidin-4-yl)-1H-benzo[d] | 0.03 |
| | imidazole-7-carboxamide | |
| 43 | N-(Azetidin-3-ylmethyl)-7-hydroxy-2-(thiophen-2-yl)- | 0.043 |
| | 1H-benzo[d]imidazole-4-carboxamide | |
| 47 | N-(4-Aminocyclohexyl)-7-hydroxy-2-(thiophen-2-yl)- | 0.051 |
| | 1H-benzo[d]imidazole-4-carboxamide | |
| 7 | 2-Cyclopropyl-4-hydroxy-N-(piperidin-2-ylmethyl)-1H- | 0.1 |
| | benzo[d]imidazole-7-carboxamide | |
| 9 | (S)-2-cyclopropyl-4-hydroxy-N-(piperidin-3-yl)-1H- | 0.1 |
| | benzo[d]imidazole-7-carboxamide | |
| 42 | 7-Hydroxy-N-(piperidin-4-ylmethyl)-2-(thiophen-2-yl)- | 0.1 |
| | 1H-benzo[d]imidazole-4-carboxamide | |
| 44 | 7-Hydroxy-N-(pyrrolidin-3-yl)-2-(thiophen-2-yl)-1H- | 0.11 |
| | benzo[d]imidazole-4-carboxamide | |
| 16 | (S)-2-Cyclopentyl-4-hydroxy-N-(piperidin-3-yl)-1H- | 0.12 |
| | benzo[d]imidazole-7-carboxamide | |
| 17 | (S)-4-Hydroxy-2-phenyl-N-(piperidin-3-yl)-1H-benzo[d] | 0.12 |
| | imidazole-7-carboxamide | |
| 15 | 2-Cyclopentyl-4-hydroxy-N-(piperidin-3-ylmethyl)-1H- | 0.13 |
| | benzo[d]imidazole-7-carboxamide | |
| 19 | 4-hydroxy-2-phenyl-N-(piperidin-3-ylmethyl)-1H-benzo | 0.13 |
| | [d]imidazole-7-carboxamide | |
| 13 | N-(azetidin-3-ylmethyl)-2-cyclopropyl-4-hydroxy-1H- | 0.14 |
| | benzo[d]imidazole-7-carboxamide | |
| 37 | (R)-7-Hydroxy-N-(piperidin-3-yl)-2-(thiophen-2-yl)-1H- | 0.18 |
| | benzo[d]imidazole-4-carboxamide | |
| 41 | 7-Hydroxy-N-(1-methylpiperidin-3-yl)-2-(thiophen-2-yl)- | 0.18 |
| | 1H-benzo[d]imidazole-4-carboxamide | |
| 11 | 2-cyclopropyl-4-hydroxy-N-(piperidin-3-yl)-1H-benzo[d] | 0.19 |
| | imidazole-7-carboxamide | |
| 12 | 2-cyclopropyl-4-hydroxy-N-(pyrrolidin-3-yl)-1H-benzo[d] | 0.21 |
| | imidazole-7-carboxamide | |
| 36 | 7-Hydroxy-N-[2-(piperazin-1-yl)ethyl]-2-(thiophen-2-yl)- | 0.21 |
| | 1H-benzo[d]imidazole-4-carboxamide | |
| 45 | 7-Hydroxy-N-(piperidin-2-ylmethyl)-2-(thiophen-2-yl)- | 0.24 |
| | 1H-benzo[d]imidazole-4-carboxamide | |
| 5 | 2-Cyclopropyl-4-hydroxy-N-(piperidin-4-ylmethyl)-1H- | 0.32 |
| | benzo[d]imidazole-7-carboxamide | |
| 8 | 2-cyclopropyl-4-hydroxy-N-(1-methylpiperidin-3-yl)-1H- | 1.3 |
| | benzo[d]imidazole-7-carboxamide | |
| 14 | 2-cyclopentyl-4-hydroxy-N-(piperidin-2-ylmethyl)-1H- | 1.3 |
| | benzo[d]imidazole-7-carboxamide | |
| 18 | 4-Hydroxy-2-phenyl-N-(piperidin-2-ylmethyl)-1H-benzo | 1.5 |
| | [d]imidazole-7-carboxamide | |
| 6 | 2-Cyclopropyl-4-hydroxy-N-(piperidin-3-yl-methyl)-1H- | 1.6 |
| | benzo[d]imidazole-7-carboxamide | |
| 21 | (7-hydroxy-2-[thiophen-2-yl]-1H-benzo[d]imidazol-4-yl) | 22 |
| | (piperazin-1-yl)methanone | |
| 48 | 2-(Bicyclo[2.2.1]heptan-2-yl)-7-hydroxy-N-(piperidin-3- | 0.068 |
| | ylmethyl)-1H-benzo[d]imidazole-4-carboxamide | |
| 49 | 2-(Bicyclo[2.2.1]heptan-2-yl)-7-hydroxy-N-(piperidin-3- | 0.23 |
| | yl)-1H-benzo[d]imidazole-4-carboxamide | |
| 50 | (S)-tert-Butyl | 3.4 |
| | 3-(2-(5-bromothiophen-2-yl)-7-hydroxy-1H-benzo[d] | |
| | imidazole-4-carboxamido)piperidine-1-carboxylate | |
| 51 | (S)-2-(5-bromothiophen-2-yl)-7-hydroxy-N-(piperidin-3- | 0.016 |
| | yl)-1H-benzo[d]imidazole-4-carboxamide | |
| 52 | 2-(Bicyclo[2.2.1]heptan-2-yl)-7-hydroxy-N-((S)- | 0.15 |
| | piperidin-3-yl)-1H-benzo[d]imidazole-4-carboxamide | |
| 53 | 2-(Bicyclo[2.2.1]heptan-2-yl)-7-hydroxy-N-(adamantane- | 1.9 |
| | 3-ylamino)-1H-benzo[d]imidazole-4-carboxamide | |
| 54 | 2-(Thiophene-2-yl)-7-hydroxy-N-(adamantane-3-ylamino)- | 0.33 |
| | 1H-benzo[d]imidazole-4-carboxamide | |
| 55 | N-(3-Aminocyclohexyl)-2-(bicyclo[2.2.1]heptan-2-yl)-7- | 0.25 |
| | hydroxy-1H-benzo[d]imidazole-4-carboxamide | |
| 56 | N-{[(cis)-4-Aminocyclohexyl]methyl}-2-(bicyclo[2.2.1] | 0.24 |
| | heptan-2-yl)-7-hydroxy-1H-benzo[d]imidazole-4- | |
| | carboxamide | |
| 57 | (S)-7-hydroxy-2-(5-(piperazin-1-yl)thiophen-2-yl)-N- | 0.038 |
| | (piperidin-3-yl)-1H-benzo[d]imidazole-4-carboxamide | |
| 58 | (R)-7-hydroxy-N-(piperidin-3-ylmethyl)-2-(thiophen-2- | 0.1 |
| | ylmethyl)-1H-benzo[d]imidazole-4-carboxamide | |
| 59 | (S)-7-hydroxy-N-(piperidin-3-yl)-2-(thiophen-2-ylmethyl)- | 0.17 |
| | 1H-benzo[d]imidazole-4-carboxamide | |
| 60 | (S)-7-hydroxy-N-(piperidin-3-ylmethyl)-2-(thiophen-2- | 0.16 |
| | ylmethyl)-1H-benzo[d]imidazole-4-carboxamide | |
| 72 | 4-[7-hydroxy-2-(thiophen-2-yl)-1H-benzo[d]imidazole-4- | 0.012 |
| | carboxamido]cyclohexanecarboxylic Acid | |

### INDUSTRIAL APPLICABILITY

The present invention provides a novel 7-Hydroxy-benzoimidazole-4-yl-methanone derivative compound having GSK-3beta inhibitory effect. The compounds of the present invention may be used for pharmaceutical composition for inhibiting GSK-3beta activity in a patient suffering from a GSK-3beta dependent disease. Such pharmaceutical compositions are suitable for treating or preventing Alzheimer disease, mania, depression, migraine and type 2 diabetes.

1. A GSK-3beta inhibitor comprising at least one compound, which is represented by formula (I), or a salt, hydrate, solvate, or isomer thereof: 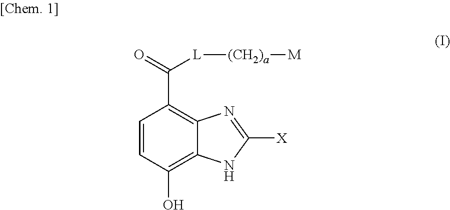
wherein
X is phenyl, thiophen-2-yl, furan-2-yl, cyclopropyl, cyclopentyl, phenyl-C₁-C₆ alkyl, thiophen-2-yl-C₁-C₆ alkyl, furan-2-yl-C₁-C₆ alkyl, cyclopropyl-C₁-C₆ alkyl cyclopentyl-C₁-C₆ alkyl, or bicyclo[2.2.1]heptan-2-yl, wherein each group is optionally substituted by 1-3 substituent(s) each independently selected from group A;
L is —NH—, or a single bond;
M is C₃-C₈ cycloalkyl, or 3-8 membered saturated heterocyclic group,
each optionally substituted by 1-3 substituent(s) each independently selected from group A;
wherein group A is selected from a group consisting of hydroxyl, oxo, nitro, cyano, amino, C₁-C₆ alkylamino, C₃-C₁₀ cycloalkylamino, amide, halogen, sulfamoyl, trifluoromethyl, p-toluenesulfonylamino, C₁-C₆ alkyl, C₃-C₈ cycloalkyl, C₁-C₆ alkoxy, C₁-C₆ alkoxycarbonyl, C₁-C₆ alkylcarbonylamino, C₁-C₆ alkylsulfonyl, C₁-C₆ alkylsulfonylamino, C₁-C₆ alkenyl, C₁-C₆ alkynyl, phosphoryl, carbonyl, carboxyl, and a 3-8 membered saturated heterocyclic group; and
a is an integer from 0 to 5.

2. The GSK-3beta inhibitor of claim 1, wherein M is piperidin-4-yl, piperidin-3-yl, piperidin-2-yl, piperazin-1-yl, pyrrolidin-3-yl, azetidin-3-yl, cyclohexyl, or adamantan-3-yl, which are each optionally substituted by 1 or 2 substituent(s) each independently selected from group A.

3. The GSK-3beta inhibitor of claim 1, wherein X is thiophen-2-yl.

4. The GSK-3beta inhibitor of claim 1, wherein X is phenyl.

5. The GSK-3beta inhibitor of claim 1, wherein X is cyclopropyl.

6. The GSK-3beta inhibitor of claim 1, wherein X is cyclopentyl.

7. The compound of claim 1, wherein X is bicycle[2.2.1]heptan-2-yl.

8. The compound of claim 1, wherein X is 5-bromothiophen-2-yl.

9. The compound of claim 1, wherein X is 5-(piperazin-1-yl)thiophen-2-yl.

10. The compound of claim 1, wherein X is thiophen-2-ylmethyl.

11. The GSK-3beta inhibitor of claim 1, which is selected from a group consisting of:
2-Cyclopropyl-4-hydroxy-N-(piperidin-4-ylmethyl)-1H-benzo[d]imidazole-7-carboxamide, 2-Cyclopropyl-4-hydroxy-N-(piperidin-3-yl-methyl)-1H-benzo[d]imidazole-7-carboxamide, 2-Cyclopropyl-4-hydroxy-N-(piperidin-2-ylmethyl)-1H-benzo[d]imidazole-7-carboxamide, 2-cyclopropyl-4-hydroxy-N-(1-methylpiperidin-3-yl)-1H-benzo[d]imidazole-7-carboxamide, (S)-2-cyclopropyl-4-hydroxy-N-(piperidin-3-yl)-1H-benzo[d]imidazole-7-carboxamide, 2-cyclopropyl-4-hydroxy-N-(piperidin-4-yl)-1H-benzo[d]imidazole-7-carboxamide, 2-cyclopropyl-4-hydroxy-N-(piperidin-3-yl)-1H-benzo[d]imidazole-7-carboxamide, 2-cyclopropyl-4-hydroxy-N-(pyrrolidin-3-yl)-1H-benzo[d]imidazole-7-carboxamide, N-(azetidin-3-ylmethyl)-2-cyclopropyl-4-hydroxy-1H-benzo[d]imidazole-7-carboxamide, 2-cyclopentyl-4-hydroxy-N-(piperidin-2-ylmethyl)-1H-benzo[d]imidazole-7-carboxamide, 2-Cyclopentyl-4-hydroxy-N-(piperidin-3-ylmethyl)-1H-benzo[d]imidazole-7-carboxamide, (S)-2-Cyclopentyl-4-hydroxy-N-(piperidin-3-yl)-1H-benzo[d]imidazole-7-carboxamide, (S)-4-Hydroxy-2-phenyl-N-(piperidin-3-yl)-1H-benzo[d]imidazole-7-carboxamide, 4-Hydroxy-2-phenyl-N-(piperidin-2-ylmethyl)-1H-benzo[d]imidazole-7-carboxamide, 4-Hydroxy-2-phenyl-N-(piperidin-3-ylmethyl)-1H-benzo[d]imidazole-7-carboxamide, 7-Hydroxy-N-(4-hydroxycyclohexyl)-2-(thiophen-2-yl)-1H-benzo[d]imidazole-4-carboxamide, (7-hydroxy-2-[thiophen-2-yl]-1H-benzo[d]imidazol-4-yl)(piperazin-1-yl)methanone, 7-Hydroxy-N-(piperidin-3-yl)-2-(thiophen-2-yl)-1H-benzo[d]imidazole-4-carboxamide, 7-Hydroxy-N-[2-(piperazin-1-yl)ethyl]-2-(thiophen-2-yl)-1H-benzo[d]imidazole-4-carboxamide, (R)-7-Hydroxy-N-(piperidin-3-yl)-2-(thiophen-2-yl)-1H-benzo[d]imidazole-4-carboxamide, (S)-7-Hydroxy-N-(piperidin-3-yl)-2-(thiophen-2-yl)-1H-benzo[d]imidazole-4-carboxamide, 7-Hydroxy-N-(piperidin-3-ylmethyl)-2-(thiophen-2-yl)-1H-benzo[d]imidazole-4-carboxamide, 7-Hydroxy-N-(piperidin-4-yl)-2-(thiophen-2-yl)-1H-benzo[d]imidazole-4-carboxamide, 7-Hydroxy-N-(1-methylpiperidin-3-yl)-2-(thiophen-2-yl)-1H-benzo[d]imidazole-4-carboxamide, 7-Hydroxy-N-(piperidin-4-ylmethyl)-2-(thiophen-2-yl)-1H-benzo[d]imidazole-4-carboxamide, N-(Azetidin-3-ylmethyl)-7-hydroxy-2-(thiophen-2-yl)-1H-benzo[d]imidazole-4-carboxamide, 7-Hydroxy-N-(pyrrolidin-3-yl)-2-(thiophen-2-yl)-1H-benzo[d]imidazole-4-carboxamide, 7-Hydroxy-N-(piperidin-2-ylmethyl)-2-(thiophen-2-yl)-1H-benzo[d]imidazole-4-carboxamide, 7-Hydroxy-N-(pyrrolidin-3-ylmethyl)-2-(thiophen-2-yl)-1H-benzo[d]imidazole-4-carboxamide, N-(4-Aminocyclohexyl)-7-hydroxy-2-(thiophen-2-yl)-1H-benzo[d]imidazole-4-carboxamide, 2-(Bicyclo[2.2.1]heptan-2-yl)-7-hydroxy-N-(piperidin-3-ylmethyl)-1H-benzo[d]imidazole-4-carboxamide, 2-(Bicyclo[2.2.1]heptan-2-yl)-7-hydroxy-N-(piperidin-3-yl)-1H-benzo[d]imidazole-4-carboxamide, (S)-tert-Butyl 3-(2-(5-bromothiophen-2-yl)-7-hydroxy-1H-benzo[d]imidazole-4-carboxamido)piperidine-1-carboxylate, (S)-2-(5-bromothiophen-2-yl)-7-hydroxy-N-(piperidin-3-yl)-1H-benzo[d]imidazole-4-carboxamide, 2-(Bicyclo[2.2.1]heptan-2-yl)-7-hydroxy-N-((S)-piperidin-3-yl)-1H-benzo[d]imidazole-4-carboxamide, 2-(Bicyclo[2.2.1]heptan-2-yl)-7-hydroxy-N-(adamantane-3-ylamino)-1H-benzo[d]imidazole-4-carboxamide, 2-(Thiophene-2-yl)-7-hydroxy-N-(adamantate adamantane-3-ylamino)-1H-benzo[d]imidazole-4-carboxamide), N-(3-Aminocyclohexyl)-2-(bicyclo[2.2.1]heptan-2-yl)-7-hydroxy-1H-benzo[d]imidazole-4-carboxamide, N-{[(cis)-4-Aminocyclohexyl]methyl}-2-(bicyclo[2.2.1]heptan-2-yl)-7-hydroxy-1H-benzo[d]imidazole-4-carboxamide, (S)-7-hydroxy-2-(5-(piperazin-1-yl)thiophen-2-yl)-N-(piperidin-3-yl)-1H-benzo[d]imidazole-4-carboxamide, (R)-7-hydroxy-N-(piperidin-3-ylmethyl)-2-(thiophen-2-ylmethyl)-1H-benzo[d]imidazole-4-carboxamide, (S)-7-hydroxy-N-(piperidin-3-yl)-2-(thiophen-2-ylmethyl)-1H-benzo[d]imidazole-4-carboxamide, (S)-7-hydroxy-N-(piperidin-3-ylmethyl)-2-(thiophen-2-ylmethyl)-1H-benzo[d]imidazole-4-carboxamide, and 4-[7-hydroxy-2-(thiophen-2-yl)-1H-benzo[d]imidazole-4-carboxamido]cyclohexanecarboxylic acid.

12. A pharmaceutical composition comprising at least one compound and pharmaceutically acceptable carrier, which is available for preventing or treating GSK-3beta dependent diseases, wherein the compound is the GSK-3beta inhibitor of claim 1.

13. The pharmaceutical composition of claim 13, wherein the GSK-3beta dependent disease is selected from a group consisting of Alzheimer disease, mania, depression, migraine and type 2 diabetes.

14. A method for treating or preventing GSK-3beta dependent diseases in a subject, comprising administering to a subject an effective amount of one compound selected from the GSK-3beta inhibitor of claim 1.

15. Use of the GSK-3beta inhibitor of claim 1 in manufacturing a pharmaceutical composition for treating or preventing a GSK-3beta dependent disease.

16. A pharmaceutical composition comprising at least one compound and pharmaceutically acceptable carrier, which is available for preventing or treating GSK-3beta dependent diseases, wherein the compound is the GSK-3beta inhibitor of claim 11.

17. The pharmaceutical composition of claim 16, wherein the GSK-3beta dependent disease is selected from a group consisting of Alzheimer disease, mania, depression, migraine and type 2 diabetes.

18. A method for treating or preventing GSK-3beta dependent diseases in a subject, comprising administering to a subject an effective amount of one compound selected from the GSK-3beta inhibitor of claim 11.

19. Use of the GSK-3beta inhibitor of claim 11 in manufacturing a pharmaceutical composition for treating or preventing a GSK-3beta dependent disease.
